(19) United States
(12) Patent Application Publication
(10) Pub. No.: US 2015/0018383 A1
(43) Pub. Date: Jan. 15, 2015

(54) BIOMARKERS OF RENAL DISORDERS

(71) Applicant: Institut National de la Sante et de la Recherche Medicale (INSERM), Paris (FR)

(72) Inventors: Fabiola Terzi, Paris (FR); Guillaume Canaud, Paris (FR); Franck Ename, Paris (FR)

(73) Assignee: Institut National de la Sante et de la Recherche Medicale (INSERM), Paris (FR)

(21) Appl. No.: 14/351,582

(22) PCT Filed: Oct. 12, 2012
(86) PCT No.: PCT/EP2012/070330

§ 371 Date: Apr. 14, 2014

(30) Foreign Application Priority Data
Oct. 14, 2011 (EP) 11185175.4

(51) Int. Cl. G01N 33/573; A61K 31/436

(52) U.S. Cl. CPC G01N 33/573; A61K 31/436; G01N 2800/52; G01N 2333/91205
USPC 514/291; 435/7.4

(57) ABSTRACT

The present invention is directed to the use of marker for Akt2 activation in podocytes as a biomarker to predict toxicity of mTOR inhibitors. Another aspect of the invention relates to a method for preventing graft rejection comprising administering a selective mTORC1 or Akt1 inhibitor in a subject in need thereof.

### FIELD OF THE INVENTION

The present invention is directed to the use of marker for Akt2 activation in podocytes as a biomarker to predict toxicity of mTOR inhibitors. Another aspect of the invention relates to a method for preventing graft rejection comprising administering a selective mTORC1 or Akt1 inhibitor in a subject in need thereof.

### BACKGROUND OF THE INVENTION

Chronic kidney disease (CKD) represents a worldwide health concern: in the United States, 20 million patients suffer from CKD and this disease is the ninth leading cause of death in the country^{1,2}. Similar rates were found in developing countries³. CKD was recently recognized as an important risk factor for death and cardiovascular diseases⁴. Understanding the pathophysiology of CKD progression is, therefore, a key challenge for medical planning.

Regardless of the initial insult, human CKD is characterized by progressive destruction of renal parenchyma and loss of functional nephrons, which ultimately lead to end stage renal failure⁵. The mechanisms of CKD progression are poorly understood, but it has been suggested that compensatory processes may play a role. In fact, a reduction in the number of functional nephrons triggers molecular and cellular events promoting compensatory growth of remaining nephrons and this compensatory process is required to maintain kidney function⁵. In glomeruli, the increase of blood flow and intracapillary pressure leads to a higher single nephron glomerular filtration rate (GFR). In tubules, the increase of transport and metabolic activities leads to higher solute and water reabsorption. However, over time, the strain imposed by these adaptations results in mechanical and metabolic stresses of the remaining nephrons that ultimately lead to nephron damage, albuminuria and further nephron loss^{5,6}. The result is a vicious cycle in which the loss of damaged nephrons leads to the damage of the so far healthy nephrons (overload hypothesis)⁷. Hence, the rate of CKD progression entirely depends on the ability of remaining nephrons to cope with stress.

Akt proteins have been shown to play a role in adaptation to physiopathological stresses^{8,9}. Akts are conserved cytosolic serine/threonine kinases that regulate many cellular stress-induced processes including survival, proliferation, migration and cytoskeletal organization¹⁰. In mammals, three distinct genes encode Akt homologs: Akt1, Akt2 and Akt3, respectively. Akt activation first requires its recruitment to the plasma membrane, which is initiated by phosphatidyl-inositol-3-kinase (PI3K), then the phosphorylation on Thr³⁰⁸ and Ser⁴⁷³ by 3-phosphoinositide-dependent protein kinase-1 (PDK1) and mammalian target of rapamycin (mTOR) complex 2 (mTORC2), respectively¹⁰. Once activated, Akt proteins phosphorylate several substrates distributed within the cell to regulate multiple cellular functions¹⁰. In the heart, the Akt signaling pathway has been shown to mediate the adaptive response to increased hemodynamic load¹¹. Similarly, the Akt pathway has been implicated in the adaptation of skeletal muscle to situations of altered use such as strength training, aging or immobilization¹².

The very few studies that have analyzed Akt activation in CKD using in vivo experimental models have led to conflicting results, indicating that Akt may act in a context- and/or cell-dependent manner^{13,14}. Indirect evidence suggests that Akt might play a role in podocyte physiology¹⁵. Podocytes are terminally differentiated, highly specialized epithelial cells that, together with endothelial cells and the glomerular basement membrane (GBM), compose the glomerular filtration barrier^{16,17}. The filtration barrier ensures that albumin and other plasma proteins are retained in the circulation. Podocytes help prevent proteinuria through a complex regulation of the actin cytoskeleton of their foot process. Interestingly, in vitro, Akt can be activated by nephrin^{18,19}, a protein of the slit diaphragm that mediates actin reorganization under mechanical stress¹⁵.

Remarkably, the inventors used experimental models of nephron reduction and Akt1 and Akt2 knockout mice to uncover a novel function of Akt2 and to show that Akt2 has a unique role in podocyte adaptation to nephron reduction. Furthermore, the inventors provided evidence that these findings are relevant to human CKD and propose a possible novel strategy to prevent the adverse renal effect of mTOR inhibitors such as sirolimus, one of the most widely used immunosuppressive drugs.

### SUMMARY OF THE INVENTION

The present disclosure demonstrates that Akt2 activation may be used to predict the adverse effect of mTOR inhibitors, such as sirolimus, on renal function or to identify patients with the highest risk to develop proteinuria, or as a therapeutic target for the maintenance of glomerular functions during chronic renal disease.

Furthermore, the present disclosure provides evidence that selective mTORC1 or Akt1 targeting therapies could be valuable alternative strategy to the non selective mTOR inhibitors to overcome the side effect in patient with reduced renal function.

Thus, in one aspect, the invention provides an in vitro method for predicting the toxicity of a treatment with mTOR inhibitors in a patient, said method comprising the steps of:
(i) providing a tissue sample previously collected from the patient to be tested;
(ii) determining, in said tissue sample, the activation level of Akt2 pathway;
wherein activation of Akt2 pathway is indicative of a likelihood of toxicity of a treatment with mTOR inhibitors in said patient.

In yet another aspect, the invention provides selective mTORC1 or Akt1 inhibitor, for use as an immunosuppressant drug in a subject in need thereof, in particular for preventing transplant rejection, such as graft kidney rejection.

### DETAILED DESCRIPTION OF THE INVENTION

### Akt2 in Podocytes as a Biomarker

The inventors have shown that Akt2 but not Akt1 in podocytes provides a stress-responsive axis in podocyte biology that prevents cytoskeleton alteration and apoptosis after nephron reduction. mTOR inhibitors such as sirolimus are widely used as immunosuppressive drugs, however, a major problem encountered with the broad use of mTOR inhibitors is their adverse effect on the kidney, particularly in patients with compromised renal function.

Therefore, the present invention provides novel biomarkers that can be used to predict the toxicity of mTOR inhibitors on the kidney, or for the prognosis of patients suffering from chronic kidney disorders.

As used herein, the term “marker” or “biomarker” refers to a molecule (typically a protein, nucleic acid, carbohydrate or lipid) that is expressed in the cell, for example Akt2 in podocyte cells, which may have detectable variable forms in vivo (for example phosphorylated or non-phosphorylated Akt2 protein) or quantifiable variable level of expression in vivo (for example Rictor protein level).

As used herein, the term “toxicity” refers to any adverse effect that can be observed and that is due directly or indirectly to the drug, for example, the mTOR inhibitor, that is administered to the patient. For example, one adverse effect of the mTOR inhibitors consist in the development of glomerular proteinuria.

As used herein, the term “mTOR inhibitor” refers to any compound capable of inhibiting at least mTORC1 complex, thus inhibiting Akt pathway. Examples of such mTOR inhibitors include, without limitation, rapamycin, sirolimus, deforolimus, temsirolimus, and everolimus or their derivatives. They further include novel dual inhibitors of mTORC1 and mTORC2 such as AZD-8055 (AstraZeneca plc), OSI-027 (OSI Pharmaceuticals Inc), INK-128, WYE-132 (Pfizer Inc) and Torin1. Other known mTOR inhibitors include without limitation ABT578, TAFA-93, PP242, Ku-0063794, biolimus-7 and biolimus-9, or AP23573.

As used herein, the term “chronic kidney disorders” or “CKD” also known as chronic renal diseases, refers to disorders with progressive loss in renal function over a period of months or years. Several pathologies may induce CKD, such as hypertension or diabete.

In the present invention, it is proposed to use Akt2 as a biomarker. Akt proteins have been shown to play a role in adaptation to physiopathological stresses. Akts are conserved cytosolic serine/threonine kinases that regulate many cellular stress-induced processes including survival, proliferation, migration and cytoskeletal organization. In mammals, three distinct genes encode Akt homologues: Akt1, Akt2 and Akt3, respectively.

In one specific embodiment, Akt2 refers to the human protein having the amino acid sequence as shown in accession number P31751 (Uniprot database) or its natural variants.

Akt activation first requires its recruitment to the plasma membrane, which is initiated by phosphatidyl-inositol-3-kinase (PI3K), then the phosphorylation on Thr³⁰⁸ and Ser⁴⁷³ by 3-phosphoinositide-dependent protein kinase-1 (PDK1) and mammalian target of rapamycin (mTOR) complex 2 (mTORC2), respectively.

In one preferred embodiment, the amount of phosphorylated (activated) Akt2 protein in podocytes is used as a biomarker. In another preferred embodiment, the amount of Rictor protein is used as a biomarker. Rictor is a member of mTORC2 complex protein, and in one specific embodiment, the term Rictor protein refers to the human protein having the amino acid sequence as shown in accession number Q6R327 (Uniprot database) or its natural variants.

Thus, in one aspect, the invention provides an in vitro method for predicting the toxicity of a treatment with mTOR inhibitors in a patient, said method comprising the steps of:
(i) providing a tissue sample previously collected from the patient to be tested;
(ii) determining, in said tissue sample, the activation level of Akt2 pathway;
(iii) comparing said activation level in said tissue sample with the activation level in a control tissue.

In one specific embodiment, activation of Akt2 pathway is indicative of a likelihood of toxicity of a treatment with mTOR inhibitors in said patient.

In another specific embodiment, a significant higher activation level of Akt2 pathway in the tested sample as compared to a control sample is indicative of a likelihood of toxicity of a treatment with mTOR inhibitors in said patient.

The term “tissue sample” includes sections of tissues such as biopsy or autopsy samples and frozen sections taken for histological purposes. Such samples include blood and blood fractions, sputum, hair, nails, skin tissue, lymph and tongue tissue, cultured cells, e.g. primary cultures, explants, stool, urine, etc.

In the methods of the invention, the tissue sample is for example a biopsy of kidney tissue, preferably kidney tissue comprising glomeruli.

The biopsy technique will depend on the tissue type to be evaluated, the size and the type of cells to analyse, among other factors. Representative biopsy techniques include, but are not limited to, excisional biopsy, needle biopsy or surgical biopsy.

At step (ii) of the methods according to the invention, the activation level of Akt2 pathway is determined. Determination of the activation level of Akt2 pathway includes the detection and/or the quantification of an activated form of a component of the Akt2 pathway in the tissue sample, and, optionally, as a control, the detection and/or the quantification of the amount of the corresponding component that is produced in a control tissue sample.

The term “Akt2 pathway” refers to Akt2 protein and the downstream components of the Akt2 pathway that are directly or indirectly activated in response to Akt2 activation. Other downstream components may include PKCalpha or SGK1. In one specific embodiment, said downstream components of the Akt2 pathway are selected among components of the Akt2 pathway which are not activated by the Akt1 pathway (Akt2-specific downstream components). Any techniques for the detection and/or the quantification of an activated component of Akt2 pathway may be used in the methods of the invention. Preferred techniques involve analysis at the protein level. Preferred techniques for analyzing and quantifying proteins in a tissue sample include western blots, immunohistochemical techniques, ELISA and mass spectrometry. Other methods involve analysis of the DNA or RNA of the tissue sample. Means for analyzing DNA or RNA include PCR, sequencing techniques, Southern Blot, Northern Blot, DNA microarray techniques, etc.

For example, immunochemical methods can be used for quantifying activated components of Akt2 pathway, such as phosphorylated (activated) Akt2 protein level, comprising in situ immunohistochemical methods on the tissue sample, for example using antibodies directed specifically against activated proteins, such as antibodies specific for P-Akt (Ser⁴⁷³) or P-Akt2 (Ser⁴⁷³). These methods are known in the art and for example described in the Examples below.

### Analysis of Proteins

In one embodiment, antibody reagents can be used to detect expression levels of proteins used as a biomarker in the methods of the invention, using any of the number of immunoassay known to those skilled in the Art. The term immunoassay encompass techniques including without limitation, enzyme immunoassays (EIA) such as enzyme multiplied immunoassay technique (EMIT), enzyme-linked immunosorbent assay (ELISA), IgM antibody capture ELISA (MAC ELISA), and microparticle enzyme immunoassay (MEIA), capillary electrophoresis immunoassays (CEIA), radioimmunoassay (RIA), immunoradiometric assays (IRMA), fluorescence polarization immunoassays (FPIA) and chemiluminescence assays (CL).

Specific immunological binding of the antibody to the protein can be detected directly or indirectly. Direct labels include fluorescent or luminescent tags, metals, dyes, radionuclides, and the like, attached to the antibody.

A signal from direct or indirect labeling can be then analysed, for example, using a spectrophotometer to detect color from a chromogenic substrate, a radiation counter to detect radiation, or a fluorometer to detect fluorescence in the presence of light of a certain wavelength.

The antibodies for use in the methods of the invention to detect the biomarkers can be immobilized onto a variety of solid supports, such as polystyrene beads, magnetic or chromatographic matrix particles, the surface of an assay plate, glass or nylon supports.

Immunohistochemical (IHC) methods, which are well known by those skilled in the art, may be used.

In one embodiment, the amount of a marker such as activated Akt2 protein or Rictor protein according to the present invention may be expressed as any arbitrary unit that reflects the amount of the corresponding P-Akt2 or Rictor protein that has been detected in the tissue sample, such as, for example, intensity of a radioactive or of a fluorescence signal emitted by the antibody material against the P-Akt protein or Rictor protein of the tissue sample.

Preferably, in one embodiment, said activation level may be expressed as any arbitrary unit that reflects the amount of the activated Akt component such as P-Akt2 (Ser⁴⁷³) or Rictor protein that has been detected in the tissue sample, such as intensity of a radioactive or of a fluorescence signal emitted by a labelled antibody specifically bound to the protein of interest. Alternatively, the value obtained at the end of step (ii) may consist of a concentration of protein(s) of interest, such as P-Akt2 (Ser⁴⁷³) or Rictor protein, that could be measured by various protein detection methods well known in the art, such as ELISA, SELDI-TOF, FACS or Western blotting.

As shown in the Examples below, Akt2 phosphorylation is barely detectable in kidney transplant biopsies from patients with preserved renal function. To the contrary, a strong staining was found in podocytes of patients with severe nephron reduction. Besides, Sirolimus administration prevented the activation of Akt in patients with impaired renal function.

In another embodiment, a level of activation is considered “high” when said level is significantly higher than the normal level observed in a control sample, said control being obtained, for example, from a subject known not to present CKD, or a healthy tissue or healthy subject.

In one embodiment, the activation level of Akt2 pathway is determined by detecting the presence of phosphorylated Akt2, for example, phosphorlylated Akt2 (Ser⁴⁷³) protein in a tissue sample comprising podocyte. In one specific embodiment, a level of activation is considered “high” when the amount of Ser⁴⁷³ Akt2 is, at least detectable, using for example specific antibodies against phosphorylated Akt2, or for example at least, twice higher in the tested tissue sample than in the control tissue sample. Amount of the biomarker in the control tissue and the tested tissue may be normalized for example with the level of a protein that is known to be constitutively and equally expressed in the tested and the control tissue.

As intended herein, a “prediction of the toxicity” does not consist of an absolute value, but it may consist of a relative value allowing quantifying the probability of risk of adverse effect, in a patient. In certain embodiments, the prediction is expressed as a statistical value, including a P value, as calculated from the expression values obtained for Akt2 activated forms and, optionally, one or more other biological markers that have been tested.

The invention further provides a method for selecting a patient susceptible to respond to a mTOR inhibitor treatment with low risk of adverse effect, said method comprising the steps of:
(i) providing a tissue sample previously collected from the patient to be tested;
(ii) determining, in said tissue sample, the activation level of Akt2 pathway;
(iii) selecting the patients with a no or low level of activation of Akt2 pathway, wherein said patient is susceptible to respond to a mTOR inhibitor treatment with low risk of adverse effect.

The invention also relates to a method for treating a patient in need of an immunosuppressant treatment, said method comprising,
(i) providing a tissue sample previously collected from the patient to be treated;
(ii) determining, in said tissue sample, the activation level of Akt2 pathway;
(iii) treating the patients with a no or low level of activation of Akt2 pathway with a mTOR inhibitor treatment and/or treating the patients with high level of activation of Akt2 pathway with a non-mTOR inhibitor immunosuppressant drug.

As used herein, the term “non-mTOR inhibitor” refers to an immunosuppressant drug that does not target mTOR pathway, such as for example, cyclosporine.

Steps (i) and (ii) of both above methods are carried out essentially as described above for the prediction methods of the invention.

As used herein, in one specific embodiment, a patient with a “normal or low” level of activation of Akt2 pathway, is a patient that has not a “high” level of activation as determined as specified in the above paragraphs, for example a patient which shows no detectable level of phosphorylated Akt2.

The invention also relates to a kit for carrying out the above prediction methods of the invention.

In particular, the invention provides a kit for the in vitro prediction of the risk of toxicity of mTOR inhibitor treatment, in one or more tissue or organ in a patient (e.g. in a tissue sample previously collected from a patient as defined above).

Another object of the present invention consists of a kit for predicting the toxicity of mTOR inhibitor treatments.

Said kits according to the invention comprises a plurality of reagents, at least one is a reagent capable of binding specifically with a component of Akt pathway, for example phosphorylated Akt2 protein, such as P-Akt2 (Ser⁴⁷³) or Rictor protein.

Suitable reagents for binding with a component of Akt pathway, such as P-Akt2 (Ser⁴⁷³) protein or Rictor protein, include antibodies, antibody derivatives, antibody fragments, and the like.

In one embodiment, said kit essentially consists of one or more reagents capable of binding specifically with a P-Akt2 protein (Ser⁴⁷³) protein.

The kits of the invention may optionally comprise additional components useful for performing the methods of the invention. By way of example, the kits may comprise fluids (e.g. SSC buffer) suitable for binding an antibody with a protein with which it specifically binds, one or more sample compartments, an instructional material which describes performance of the prediction method or of the monitoring method of the invention, and the like.

More generally, the biomarkers of the invention may also be used to monitor the influence of tested compounds (e.g. drug compounds) on the renal function of a patient with time. For example, the capacity of a compound to affect Akt2 activation level can be monitored during treatments of subjects receiving such tested compounds: A higher level in the Akt2 activation is indicative that the drug or its tested dosage may be toxic for the renal function.

Method of Screening Novel Immunosuppressive Agents with Less Risk of Renal Adverse Effect

For those patients at risk of adverse effect with mTOR inhibitors, novel drugs that are capable of inhibiting selectively Akt1 pathway or mTORC1 inhibitors may be used according to the present invention.

Thus the invention also relates to methods of screening of novel immunosuppressive agent, selected from selective Akt1 and/or mTORC1 inhibitors.

In one specific embodiment, the invention provides methods for screening novel immunosuppressive agents comprising (i) optionally first selecting compounds that binds to Akt1 and/or mTORC1 with high affinity in a primary binding assay and (ii) selecting, for example from those binding compounds optionally first selected in step (i), the compounds that selectively inhibit Akt1 pathway but not Akt2 pathway in a secondary functional assay.

The screening methods of the invention may comprise a first primary binding screening assay, generally carried as a high throughput screening assay, designed to identify compounds that bind with a high affinity to a component of the Akt pathway, for example mTORC1. In one embodiment, “high affinity” refer to compounds that binds to the target, for example, mTORC1, with a dissociation constant K_{D} of 100 μM or less, 10 μM or less, 1 μM or less, 100 nM or less, 10 nM or less, or 1 nM or less. K_{D} affinity can be measured for example using surface Plasmon resonance, such as Biacore® assay.

Compounds may be tested from large libraries of small molecules, natural products, peptides, peptidomimetics, polypeptides, proteins or a combination thereof or any appropriate compound libraries for drug discovery. Synthetic compound libraries are commercially available from Maybridge Chemical Co (Trevillet, Cornwall, UK), Comgenex (Princeton, N.J.), Brandon Associates (Merrimack, N.H.), and Microsource (New Milford, Conn.).

Once hit molecules or binding compounds have been selected from the primary screening assay, they are generally subject to a secondary functional assay for testing specific and/or selective inhibition of mTORC1 or Akt1 kinase activity.

The term “inhibitors of mTORC1 or Akt1” as used herein relates to compounds capable of fully or partially preventing, or reducing or inhibiting Akt1 or mTORC1 dependent activation of the Akt pathway.

As used herein, the term “specific inhibition” refers to an inhibition that is dependent upon the presence of an agonist or natural ligand of Akt1 pathway, preferably dose-dependent. Intensity of the inhibition can be referred as IC₅₀, i.e, the concentration of the inhibitors required to obtain 50% of inhibition in a determined assay. In one embodiment, specific inhibitors have an IC₅₀ of 100 μM or less, 10 μM or less, 1 μM or less, 100 nM or less, 10 nM or less or 1 nM or less, as measured in said secondary functional assay.

Selective inhibition refers to an inhibition of Akt pathway that selectively inhibits Akt1 pathway (for example mTORC1 kinase activity) but not Akt2 pathway, (for example mTORC2 kinase activity).

Preferred compounds for use according to the invention are compound capable of inhibiting mTORC1 kinase activity with an IC50 that is at least 10 times, preferably 10² times and more preferably 10³ times lower than the IC50 for mTORC2 kinase activity. In another embodiment, preferred compounds are selective and specific inhibitors of mTORC1 that exhibit no significant inhibition of mTORC2 (i.e. an inhibition of mTORC2 kinase that is barely detectable in conventional biochemical in vitro assay or with an IC50 higher than 100 μM).

Biochemical assays for detecting inhibition of mTORC1 and/or mTORC2 kinase activities may be carried out as described below in the Examples with Akt2 target. Similarly, for detecting activation of mTORC1 pathway, phospho-4EBP, phospho-S6K may be detected using specific antibodies, and for detecting activation of mTORC2 pathway, activated PKCalpha or SGK1 may be detected using specific antibodies.

Compounds that exhibit one or more inhibition properties, the “lead” molecules, may then be chemically modified, for example for improving their binding properties, their pharmacokinetic and pharmacodynamic properties (e.g. solubility and ADME properties).

Selective Inhibitors of mTORC1 or Akt1 for Use as Immunosuppressive Agent

The invention more specifically relates to selective inhibitors of mTORC1 or Akt1, for use as drug, for example for use as immuosuppressive drug, or for preventing transplant rejection such as kidney transplant rejection.

Such inhibitors may be selected among small molecule, siRNA, shRNA, anti-sense DNA and the like.

In one embodiment, it is selected from the group consisting of siRNA, shRNA, anti-sense oligonucleotides and ribozymes.

Small inhibitory RNAs (siRNAs) can function as inhibitors of gene expression of a component of Akt1 pathway. For example, gene expression of Akt1 or a member of mTORC1 complex can be reduced by contacting a subject or cell with a small double stranded RNA (dsRNA), or a vector or construct causing the production of a small double stranded RNA, such that said gene expression of Akt1 or related mTORCI complex member is specifically inhibited (i.e. RNA interference or RNAi). Methods for selecting an appropriate dsRNA or dsRNA-encoding vector are well known in the art for genes whose sequence is known (e.g. see for example Tuschl, T. et al. Genes Dev. 1999 Dec. 15; 13(24):3191-7; Elbashir, S. M. et al Nature. 2001 May 24; 411(6836):494-8; Hannon, G J. Nature. 2002 Jul. 11; 418(6894):244-51); McManus, M T. et al. J Immunol 169, 5754-5760 (2002); Brummelkamp, T R. et al. Science. 2002 Apr. 19; 296(5567):550-3; U.S. Pat. Nos. 6,573,099 and 6,506,559; and International Patent Publication Nos. WO 01/36646, WO 99/32619, and WO 01/68836). All means and methods which result in a decrease in Akt1 gene expression or in one other gene involved in mTORC1 expression, in particular by taking advantage of Akt1-specific siRNAs (i.e siRNAs that target specifically Akt1 mRNA) may be used in the present invention. Methods for generating and preparing siRNA(s) as well as method for inhibiting the expression of a target gene are also described for example in WO02/055693.

siRNAs or related nucleic acids useful as inhibitors of Akt1 gene expression, such as anti-sense oligonucleotides can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone. Those modification includes the use of nucleosides with modified sugar moieties, including without limitation, 5′-vinyl, 5′-methyl (R or S), 4′-S, 2′-F, 2′-OCH₃ and 2′-O(CH₂)₂OCH₃ substituent groups. The substituent at the 2′ position can also be selected from allyl, amino, azido, thio, O-allyl, O—C₁-C₁₀ alkyl, OCF₃, O(CH₂)₂SCH₃, O(CH₂)₂—O—N(Rₘ)(Rₙ), and O—CH₂—C(═O)—N(Rₘ)(Rₙ), where each Rₘ and Rₙ is, independently, H or substituted or unsubstituted C₁-C₁₀ alkyl.

Antisense oligonucleotides and siRNAs or related nucleic acids useful as inhibitors of Akt1 pathway may be delivered in vivo alone or in association with a vector. In its broadest sense, a “vector” is any vehicle capable of facilitating the transfer of the antisense oligonucleotide or siRNA or related nucleic acids to the target cells, preferably those with deficient expression of SMN gene, such as muscular cells. Preferably, the vector transports the nucleic acid to cells with reduced degradation relative to the extent of degradation that would result in the absence of the vector. In general, the vectors useful in the invention include, but are not limited to, plasmids, phagemids, viruses, transposon-based vectors or other vehicles derived from viral or bacterial sources that have been manipulated by the insertion or incorporation of the antisense oligonucleotide or siRNA or related nucleic acid sequences. Viral vectors are a preferred type of vector and include, but are not limited to, nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Varmus, Harold; Coffin, John M.; Hughes, Stephen H., ed (1997). “Principles of Retroviral Vector Design”. Retroviruses. Plainview, N.Y.: Cold Spring Harbor Laboratory Press. ISBN 0-87969-571-4.

Preferred viruses for certain applications are the adeno-viruses and adeno-associated viruses or retroviral vectors such as lentiviruses, which are double-stranded DNA viruses that have already been approved for human use in gene therapy. Examples of such viral vectors includes vectors originated from retroviruses such as HIV (Human Immunodeficiency Virus), MLV (Murine Leukemia Virus), ASLV (Avian Sarcoma/Leukosis Virus), SNV (Spleen Necrosis Virus), RSV (Rous Sarcoma Virus), MMTV (Mouse Mammary Tumor Virus), etc, lentivirus, Adeno-associated viruses, and Herpes Simplex Virus, but are not limited to.

Theses viral vectors can be engineered to be replication deficient and is capable of infecting a wide range of cell types and species. It further has advantages such as, heat and lipid solvent stability; high transduction frequencies in cells of diverse lineages, including hematopoietic cells; and lack of superinfection inhibition thus allowing multiple series of transductions.

Other vectors include plasmid vector, cosmid vector, bacterial artificial chromosome (BAC) vector, transposon-based vector. Plasmids may be delivered by a variety of parenteral, mucosal and topical routes. For example, the DNA plasmid can be injected by intramuscular, eye, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

In a preferred embodiment, the antisense oligonucleotide, siRNA, shRNA or related nucleic acid sequence is under the control of a heterologous regulatory region, e.g., a heterologous promoter. The promoter can also be, e.g., a viral promoter, such as CMV promoter or any synthetic promoters.

siRNA can also be directly conjugated with a molecular entity designed to help targeted delivery. Examples of conjugates are lipophilic conjugates such as cholesterol, or aptamer-based conjugates. Cationic peptides and proteins are also used to form complexes with a negatively charged phosphate backbone of the siRNA.

### Method of Treatment and Pharmaceutical Compositions

Another object of the invention relates to the use of the selective mTORC1 or Akt1 inhibitor as an immunosuppressant drug in a subject in need thereof.

As used herein, the term “subject” refers to an animal. Typically the animal is a mammal. A subject also refers to, for example, primates (e.g., human), cows, sheep, goats, horses, dogs, cats, rabbits, rats, mice, fish, birds and the like. In one preferred embodiment, the subject is a human.

The term “treatment” refers to alleviating or ameliorating the disease or disorder or at least one of the clinical symptoms. In one specific embodiment, said term includes alleviating or ameliorating at least one physical parameter that is not discernible by the patient.

The invention further relates to methods for patients suffering from kidney disorders, for example, chronic kidney disorders, in need of an immunosuppressant treatment, said method comprising administering to a subject in need thereof a therapeutically effective amount of compound which is a selective mTORC1 or Atk1 inhibitor as described above.

In one specific embodiment, the invention relates to methods for preventing transplant rejection, such as graft kidney rejection, comprising administering to a subject in need thereof a therapeutically effective amount of compound which is a selective mTORC1 or Akt1 inhibitor as described above.

The compounds of the invention as described above may be administered in the form of a pharmaceutical composition, as defined below.

By a “therapeutically effective amount” is meant a sufficient amount of compound to treat and/or to prevent, reduce and/or alleviate one or more of the symptoms of the disease.

It will be understood that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed, the age, body weight, general health, sex and diet of the patient; the time of administration, route of administration, and rate of excretion of the specific compound employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the patient to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

Hence, the present invention also provides a pharmaceutical composition comprising an effective dose of a selective mTORC1 or Akt1 inhibitor, according to the invention.

Any therapeutic agent of the invention may be combined with pharmaceutically acceptable excipients, and optionally sustained-release matrices, such as biodegradable polymers, to form therapeutic compositions.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The form of the pharmaceutical compositions, the route of administration, the dosage and the regimen naturally depend upon the condition to be treated, the severity of the illness, the age, weight, and sex of the patient, etc.

The pharmaceutical compositions of the invention can be formulated for a topical, oral, intranasal, parenteral, intraocular, intravenous, intramuscular or subcutaneous administration and the like.

In the following, the invention will be illustrated by means of the following examples as well as the figures.

### FIGURE LEGENDS

FIG. 1: Nephron reduction activates Akt pathway in podocytes. (a) Representative frozen section of a glomerulus before and after laser capture microdissection. (b) Western blot of Akt phosphorylation of Ser⁴⁷³ on laser microdissected glomerular extracts from kidneys of sham-operated (Sh) and 75% nephrectomized (Nx) FVB/N mice, 2 months after surgery. (c) Immunostaining and quantification of Gsk3α and Gsk3β phosphorylation in Sh and Nx mice, 2 months after surgery. (d) Immunostaining showing the colocalization between P-Gsk3α and nephrin, a podocyte specific protein. (e) Immunostaining of Akt1 and Akt2 proteins in Sh and Nx FVB/N mice 2 months after surgery. Akt1^{-/-} and Akt2^{-/-} mice served as control of antibody specificity (C⁻). (f) Colocalization between Akt2 (brown staining) and WT1 (blue staining), a podocyte specific protein. Akt2^{-/-} mice served as control. Pictures are representative samples from at least 6 mice from each group. Scale bar: 50 μm. Data are means±SEM; n=4-6 and 6-10 for Sh and Nx, respectively. Mann-Whitney test: Sh versus Nx mice * P<0.05, ** P<0.01.

FIG. 2: Akt2 deficiency accelerates the development of glomerular lesions. (a) Morphology and lesion scores of kidneys from sham-operated (Sh) and 75% nephrectomized (Nx) Akt2^{+/+} and Akt2^{-/-} mice, 2 months after surgery. (b) Coomassie staining (left panel) and measure of albumin to creatinine ratio (mg/mmol) (right panel) in urine from Sh and Nx Akt2^{+/+} and Akt2^{-/-} mice, 2 months after surgery. Bovine Serum Albumin served as control (C⁺) for Coomassie staining. (c) Western blot and quantification of P-Akt (Ser⁴⁷³) in laser microdissected glomerular extracts from kidneys of Sh and Nx Akt2^{+/+} and Akt2^{-/-} mice, 2 months after surgery. (d) Western blot and quantification of Akt1 and Akt2 in laser microdissected glomerular extracts from kidneys of Sh and Nx Akt2^{+/+} and Akt2^{-/-} mice, 2 months after surgery. Pictures are representative samples from at least 6 mice from each group. Scale bar: 50 μm. Data are means±SEM; n=4-6 and 6-10 for Sh and Nx, respectively. ANOVA followed by Tukey-Kramer test; Sh versus Nx mice: * P<0.05, ** P<0.01, *** P<0.001; Akt2^{-/-} versus Akt2^{+/+} mice: # P<0.05, ## P<0.01, ### P<0.001.

FIG. 3: Akt2 regulates podocyte survival and morphology. (a) PCNA immunostaining (upper panels, black arrows) and quantification of PCNA-positive glomerular cells in kidneys from sham-operated (Sh) and 75% nephrectomized (Nx) Akt2^{+/+} and Akt2^{-/-} mice, 2 months after surgery. TUNEL assay (middle panels, white arrows) and quantification of TUNEL-positive glomerular cells in kidneys from Sh and Nx Akt2^{+/+} and Akt2^{-/-} mice, 2 months after surgery. WT1 immunostaining (lower panels) and quantification of WT1-positive glomerular cells in kidneys from Sh and Nx Akt2^{+/+} and Akt2^{-/-} mice, 2 months after surgery. Pictures are representative samples from at least 6 mice from each group. (b) TUNEL assay (upper panels, white arrows) and WT1 immunostaining (lower panels) in kidneys from Akt2^{+/+} and Akt2^{-/-} mice at 13 months of age. The graphs represent the quantification of the TUNEL-positive and the WT1-positive glomerular cells. Pictures are representative samples from at least 5 mice from each group. (c) Electron microscopy of glomerular basement membrane and quantification of foot process effacement in kidneys from Akt2^{+/+} and Akt2^{-/-} mice at 13 months of age. Akt2^{-/-} deficiency led to variable degrees of foot process effacement; in addition, even when foot processes were present, slit diaphragms were not evident. Some apoptotic podocytes were also observed in Akt2^{-/-} mice (white arrow). Pictures are representative samples from at least 4 mice from each group. Scale bar: 50 μm. Data are means±SEM; n=4-6, 6-10 and 4-5 for Sh, Nx, and 13 month-old mice, respectively. Mann-Whitney test (when two groups are compared) or ANOVA followed by Tukey-Kramer test (when more than two groups are compared). Sh versus Nx mice: * P<0.05, ** P<0.01, *** P<0.001; Akt2^{-/-} versus Akt2^{+/+} mice: # P<0.05, ## P<0.01, ### P<0.001.

FIG. 4: Akt2 controls critical targets for podocyte survival after nephron reduction. (a) Western blot and quantification of P-Mdm2 and P-Gsk3α, two downstream targets of P-Akt, in laser microdissected glomerular extracts from kidneys of sham-operated (Sh) and 75% nephrectomized (Nx) Akt2^{+/+} and Akt2^{-/-} mice, 2 months after surgery. (b) Immunostaining and quantification of Gsk3α and Gsk3β phosphorylation in kidneys from Sh and Nx Akt2^{+/+} and Akt2^{-/-} mice, 2 months after surgery. Pictures are representative samples from at least 6 mice from each group. (c) Rac1-GTP bound assay in kidneys of Sh and Nx Akt2^{+/+} and Akt2^{-/-} mice, 2 months after surgery. (d) RhoA-GTP bound assay in kidneys from Sh and Nx Akt2^{+/+} and Akt2^{-/-} mice, 2 months after surgery. Scale bar: 50 μm. Data are means±SEM; n=4-6 and 6-10 for Sh and Nx, respectively. ANOVA followed by Tukey-Kramer test; Sh versus Nx mice: * P<0.05, ** P<0.01; Akt2^{-/-} versus Akt2^{+/+} mice: # P<0.05, ## P<0.01.

FIG. 5: Akt function in podocytes is isoform specific. (a) Coomassie staining (left panel) and measure of the albumin to creatinine ratio (mg/mmol) (right panel) in C57BL/6 wild type (WT), Akt1^{-/-} and Akt2^{-/-} mice at 3 and 6 months of age. Only, the Akt2^{-/-} mice developed albuminuria. Bovine Serum Albumin served as control (C⁺) for Coomassie staining. (b) Renal morphology of kidneys from WT, Akt1^{-/-} and Akt2^{-/-} mice at 6 months of age. Whereas Akt1^{-/-} mice were indistinguishable from wild-type littermates, Akt2^{-/-} mice displayed tubular dilations with casts (asterisk). (c) Electron microscopy of glomerular basement membrane and quantification of foot process effacement in kidneys from WT, Akt1^{-/-} and Akt2^{-/-} mice at 6 months of age. Variable degrees of foot process effacement and the loss of the slit diaphragms were observed, but exclusively in kidneys from Akt2^{-/-} mice. (d) TUNEL assay (upper panels, white arrows) and WT1 immunostaining of kidneys from wild type, Akt1^{-/-} and Akt2^{-/-} mice at 6 months of age. The graphs represent the quantification of the TUNEL-positive and the WT1-positive glomerular cells. Pictures are representative samples from at least 6 mice from each group. Scale bar: 50 μm and 1 μm, in b-d and c, respectively. Data are means±SEM; n=5-11 for wild type, Akt1^{-/-} and Akt2^{-/-}mice. ANOVA followed by Tukey-Kramer test; Akt1^{-/-} and Akt2^{-/-} versus WT mice: * P<0.05; Akt2^{-/-} versus Akt1^{-/-} mice: # P<0.05.

FIG. 6: Akt phosphorylation is critical to prevent proteinuria in humans. (a) Clinical data defining the four groups of renal transplant recipients: patients with higher estimated glomerular filtration rate (eGFR) +/− sirolimus and patients with lower eGFR +/− sirolimus. (b) Glomerular immunostaining of P-AKT (Ser⁴⁷³) in the four groups of patients. Akt phosphorylation could be detected exclusively in podocytes of kidneys from renal transplant recipients with lower eGFR. Sirolimus completely blunted such activation. (c) Glomerular TUNEL assay (white arrows) in the four groups of patients. Sirolimus treatment led to a marked increased of apoptosis, but exclusively in kidneys from renal transplant recipients with lower eGFR. (d) Glomerular immunostaining of RICTOR in the four groups of patients. Paralleling P-AKT pattern, Rictor protein could be detected exclusively in podocytes of kidneys from renal transplant recipients with lower eGFR. Sirolimus completely blunted such increase. (e) Quantification of glomerular P-AKT staining (left panel), TUNEL assay (middle panel) and RICTOR staining (right panel)s. Pictures are representative samples from at least 10 patients from each group. Scale bar: 50 μm. Data are means±SEM; n=20, 22, 25 and 13 in high eGFR +/− sirolimus and low eGFR +/− sirolimus, respectively. ANOVA followed by Tukey-Kramer test. Low eGFR versus high eGFR: *** P<0.001; Siro+ versus Siro−: ### P<0.001.

FIG. 7: Inhibition of podocyte Akt phosphorylation by sirolimus parallels albuminuria development in humans. Urinary albumin excretion (lower panels) and P-Akt (Ser⁴⁷³) immunostaining (upper panels) in serial biopsies from two representative renal transplant patients, each of them was treated or not with sirolimus. Tx=transplantation. Note the strong correlation between the inhibition of Akt phosphorylation and the development of albuminuria; whereas sirolimus treatment leads to severe albuminuria and blunts Akt phosphorylation in patients 1, its removal restores Akt phosphorylation and reverses albuminuria in patient 2. Scale bar: 50 μm.

FIG. 8: Akt2 is an intrinsic regulator of podocyte cytoskeleton and function. (a) measure of the albumin to creatinine ratio (mg/mmol) in 75% nephrectomized (Nx) C57BL/6 Akt2^{flox} and Akt2^{Δpod}, 1 month after surgery. Only, Akt2^{Δpod} mice developed albuminuria. (b) Quantification of foot process effacement in kidneys from Akt2^{flox} and Akt2^{Δpod} mice 1 month after Nx. Podocyte specific Akt2 deficiency led to variable degrees of foot process effacement; in addition, even when foot processes were present, the slit diaphragm was not evident. (c) Quantification of the number of focal adhesions in podocytes transfected with Sc, Sh Akt1 or Sh Akt2 and treated or not with sirolimus. Similar results were obtained with two independent lentiviral constructs targeting each Akt isoforms. Data are means±SEM; n=16-18 for Akt2^{flox} and Akt2^{Δpod} mice, respectively, and n=3-6 for in vitro experiments. Mann-Whitney test: Akt2^{flox} versus Akt2^{Δpod} mice *** P<0.001. ANOVA followed by Tukey-Kramer test: Sh versus Sc: *** P<0.001; Sh Akt2 versus Sh Akt1: ### P<0.001, Sirolimus versus Vehicle: ^{ooo} P<0.001.

### EXAMPLES

In the following description, all molecular biology experiments for which no detailed protocol is given are performed according to standard protocol.

### Methods

### Animals

Mice used for these studies were FVB/N and C57BL/6 (Charles River), Akt1^{-/-} and Akt2^{-/-} mice^{23,38}. Akt1^{-/-} and Akt2^{-/-} mice were bred onto an FVB/N genetic background for at least 10 generations. Animals were fed ad libitum and housed at constant ambient temperature in a 12-hour light cycle. Animal procedures were approved by the Departmental Director of “Services Vétérinaires de la Préfecture de Police de Paris” and by the ethical committee of the Paris Descartes University.

Several groups of mice were investigated in complementary studies. For subototal nephrectomy studies, FVB/N Akt2^{-/-} mice were subjected to 75% nephrectomy (Nx; n=6-10) or sham-operation (Controls; n=4-6), as previously described⁵³. After surgery, mice were fed a defined diet containing 30% casein and 0.5% sodium. Mice were sacrificed 2 months after surgery. One week before sacrifice, blood pressure was recorded in both sham-operated (n=3) and subtotally nephrectomized (n=6) awake Akt2^{+/+} and Akt2^{-/-} mice for 2 consecutive days, using tail-cuff plethysmography and PowerLab/4SP software (AD Instruments). Twenty-four hours before sacrifice, blood was collected from the tail vein of overnight-fasted mice for determination of fasting glucose levels. For aging nephropathy studies on FVB/N background, 4-6 Akt2^{+/+} and Akt2^{-/-} mice were followed, respectively, and urine were collected at 2, 4, 6, 9, 12 and 13 months. Mice were sacrificed at 13 months of age. For aging nephropathy studies on C57BL/6 background, wild type, Akt1^{-/-} and Akt2^{-/-} mice were followed (n=5-11 for each genotype), and urine were collected at 3 and 6 months. Mice were sacrificed at 6 months of age. At the time of sacrifice, kidneys were removed for morphological, protein and microdissection studies.

### Clinical Samples

The study was conducted on 80 renal transplant recipients followed at the Renal Transplant Department of Necker hospital. The patients were divided into four groups: 1) patients (n=13) fulfilling the criteria of sirolimus-induced albuminuria, i.e. (i) the occurrence of a severe albuminuria (>3 g/day), (ii) no other causes of post-transplant albuminuria, and (iii) a reduction of 50% of albuminuria after sirolimus withdrawal or significant dosage reduction; 2) patients (n=25) matched for glomerular filtration rate, as estimated by MDRD formula (eGFR) who did not receive sirolimus; 3) patients (n=22) with sirolimus treatment that did not develop albuminuria; 4) patients (n=20) with preserved renal function (eGFR >60 mL/mn/1.73 m²) who did not receive sirolimus.

This protocol was approved by the Institutional Review Board of Necker Hospital; informed written consent was obtained from each patient.

### Cell Cultures

Mouse immortalized podocytes were cultured as previously described⁵⁷. Briefly, cells were maintained in RPMI 1640 medium supplemented with 10% fetal calf serum, 100 U/ml penicillin, 100 μg/ml streptomycin. To propagate podocytes, cells were cultivated at 33° C. on type I collagen (permissive conditions) in culture medium supplemented with 10 UI/ml recombinant interferon-. To induce differentiation, podocytes were maintained on type I collagen at 37° C. without supplementation with interferon- (non permissive conditions). For sirolimus experiments, cells were treated with sirolimus 10 ng/mL for 5 days before performing immunofluorescence. Each experiment was performed in duplicate and repeated at least three times.

### Lentiviral Production and Transfection

Lentiviral shRNA plasmids for Akt1 and Akt2 were obtained from Sigma-Aldrich. We used a target set of 5 clones for each target with pLKO.1-puro as backbone (TRCN0000022934, TRCN0000022935, TRCN0000022936, TRCN0000022937 and TRCN0000054707 for Akt1 and TRCN0000055258, TRCN0000055259, TRCN0000055260, TRCN0000022669 and TRCN0000022670 for Akt2). The lentiviral particles were produced by co-transfection of HEK293T cells with three plasmids (pMD2G, psPAX2, and the plasmid vector) using calcium phosphate method. Cells were infected in the presence of 8 μg/ml polybrene over night, and were selected 2 days after viral transduction in puromycin (1 μg/ml) to achieve 100% positive cells. For each targeted Akt isoform, two lentiviral constructs were selected for the subsequent experiments, according to the level and the selectivity of Akt1 or Akt2 protein reduction as evaluated by western blot.

### Urine and Plasma Analyses

For mouse samples, urinary albumin, glucose and creatinine concentrations were measured using an Olympus multiparametric analyzer (Instrumentation Laboratory). In addition, Coomassie gels were used to visualize albuminuria. Blood glucose levels were determined using a glucotrend glucometer (Roche Diagnostics).

For human samples, urinary creatinine and albumin were determined using a Hitachi 917 analyzer (Roche Diagnostics), whereas plasma creatinine and sirolimus were evaluated using a Synchron Cx4 autoanalyser (Beckman Coulter) and high-performance liquid chromatography, respectively.

### Morphological Analysis

Mouse kidneys were fixed in 4% paraformaldehyde, paraffin embedded, and 4-μm sections were stained with periodic acid Schiff (PAS), Masson's trichrome, hematoxylin and eosin (H&E) and picro-sirius red. The degree of glomerular lesions was evaluated using semiquantitative score methodology as previously described²⁰. The degree of tubular dilation and interstitial fibrosis was automatically quantified using a Nikon digital camera Dx/m/1200 and Lucia software (Laboratory Imaging Ltd) as previously described⁵⁴.

Human transplant biopsies were fixed in alcohol-formalin-acetic acid solution (AFA) and embedded in paraffin. Sections (4 μm) were stained with PAS, Masson's trichrome and H&E.

Electron microscopy studies were performed as previously described⁵⁵.

### Immunohistochemistry

For mouse samples, 4-μm sections of paraffin-embedded kidneys were incubated with anti-Akt1 antibody (Cell Signaling Technology), anti-Akt2 antibody (Cell Signaling Technology), anti-pan-P-Gsk3 antibody (Cell Signaling Technology), anti-P-Gsk3α antibody (Cell Signaling Technology), anti-P-Gsk3β antibody (Cell Signaling Technology), anti-P-Mdm2 antibody (Cell Signaling Technology), anti-Tsc2 antibody (Cell Signaling Technology), anti-Foxo1 antibody (Cell Signaling Technology), anti-Nephrin (Progen), anti-WT1 antibody (Dako), anti-podocin antibody (a gift of Corinne Antignac) and anti-αSma (Sigma, A2547). For P-Gsk3α and P-Gsk3β the glomerular-stained area was automatically quantified using a Nikon digital camera Dx/m/1200 and Lucia software and expressed as the percentage of the podocyte-stained area upon the total glomerular area. To quantify the hypertrophy of renal large and small arteries, the αSma stained area was automatically quantified for each vessel using the same system described above. All the microscopic fields of a whole kidney section were quantified.

For human samples, 4-μm sections of paraffin-embedded kidneys were incubated with P-Akt Ser⁴⁷³ antibody (Cell Signaling Technology), anti-Rictor antibody (Cell Signaling Technology), anti-P-S6RP antibody (Cell Signaling Technology) and anti-P-4EBP1 antibody (Cell Signaling Technology). For each antibody, the intensity of the staining was determined using a semiquantitative score methodology based on a 0-2 staining scale; 0, 1 and 2 correspond to 0%, 1-50% and >50% of positive-podocytes for glomerulus. All the glomeruli of a single section were counted.

### Cell Proliferation Assay

Proliferative cells were detected in mouse kidney using proliferating cell nuclear antigen (PCNA) immunostaining. Four-μm sections of paraffin-embedded kidneys were incubated with a mouse anti-PCNA antibody (DAKO) at 1:50, followed by a sheep HRP-conjugated anti-mouse antibody (Amersham) at 1:100. Staining was revealed by DAB. The glomerular proliferation index was calculated as the number of glomeruli with at least one PCNA-positive nuclei for the total number of glomeruli. All the microscopic fields of a whole kidney section were quantified.

### Apoptosis Assay

Apoptosis was detected in 4-μm sections of paraffin-embedded kidneys by TUNEL assay using the In Situ Cell Death Detection kit (Roche) according to the manufacturer's protocol. The glomerular apoptotic index was calculated as the number of glomeruli with at least one TUNEL-positive nuclei for the total number of glomeruli. All the microscopic fields of a whole kidney section were quantified.

### Isolation of Adult Mice Glomeruli by Laser Capture Microdissection

Thirty micrometer frozen kidney sections were fixed in absolute ethanol and stained with eosin Y. Glomeruli were selectively dissected using the Palm MicroBeam laser microdissection microscope (Zeiss). A total of 1200 glomerular structures were microdissected from each mouse and proteins were extracted with the appropriate buffer.

### Western Blot

Western blots were performed as previously described^{54,56}. Briefly, protein extracts from kidneys was resolved by SDS-PAGE before being transferred onto the appropriate membrane and incubated with anti-P-Akt Ser⁴⁷³ antibody (Cell Signaling Technology, 4060), anti-Akt1 antibody (Cell Signaling Technology, 2938), anti-Akt2 antibody (Cell Signaling Technology, 3063), anti-P-Gsk3α antibody (Cell Signaling Technology, 2938), anti-P-Mdm2 antibody (Cell Signaling Technology), anti-Rac1 antibody (Upstate Biotechnology), anti-RhoA antibody (Cytoskeleton) and anti-β actin antibody (Sigma-Aldrich, A5316), followed by the appropriate peroxidase-conjugated secondary antibody. Chemiluminescence was acquired using a Fusion FX7 camera (Vilbert Lourmat) and densitometry was performed using Bio1D software (Certain Tech).

### Rho GTPase Activation Assay

RhoA activity was measured using a commercially available kit (Cytoskeleton) according to the manufacture's instructions. For Rac1 activity, a PAK1-GST fusion protein expressing vector (a gift of Rodrick Montjean) was used to transform the BL21 strain of Escherichia coli. The fusion protein was purified with glutathione-Sepharose 4B beads (Amersham). The beads where incubated with kidney lysate for 1 hour at 4° C. Then the beads were washed in lysis buffer and the pulled down fraction of Rac1 was resolved by immunoelectrophoresis.

### Data Analysis and Statistics

Data were expressed as means±SEM. Differences between the experimental groups were evaluated using ANOVA, followed when significant (P<0.05) by the Tukey-Kramer test. When only two groups were compared, Mann-Whitney tests were used. The statistical analysis was performed using Graph Prism Software.

### Results

### Nephron Reduction Activates Akt Pathway in Podocytes

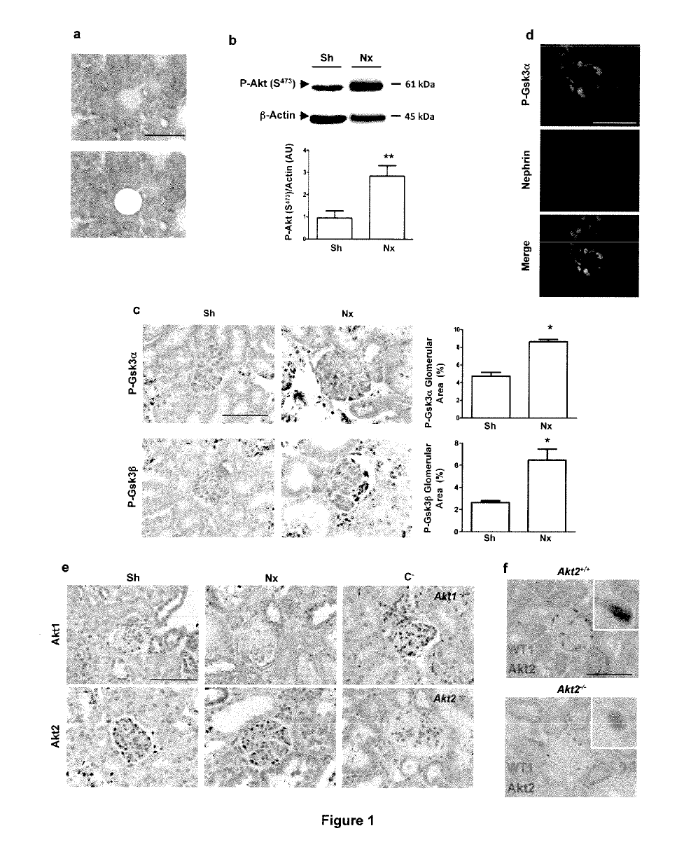

To investigate whether Akt activation might be involved in adaptation to the stress imposed by nephron loss during CKD, we performed 75% nephron reduction in lesion-prone FVB/N mice²⁰ and analyzed Akt phosphorylation. Western blot analysis revealed that neither Akt nor its downstream target Gsk3 were phosphorylated in whole kidney extracts two months after nephron reduction (data not shown). However, on laser capture microdissected glomeruli (FIG. 1a), we found a robust (3-fold) increase in Akt phosphorylation at residue Ser⁴⁷³ (FIG. 1b). Immunohistochemical analysis of P-Gsk3α and P-Gsk3β confirmed the activation of the pathway in glomeruli (FIG. 1c). More importantly, it revealed that P-Gsk3α and P-Gsk3β are predominantly expressed in outlying glomerular cells with the typical morphologic features of podocytes. Colocalization experiments using an antibody directed against nephrin, a podocyte specific protein, corroborated these observations (FIG. 1d). In addition, we observed that three other Akt targets (Mdm2, Tsc2, Foxo-1) were phosphorylated in podocytes (data not shown). Hence, Akt activation after nephron reduction seems to be compartmentalized in podocytes.

### Akt2 Isoform is Specifically Enriched in Podocytes

It is known that Akt1 and 2, but not Akt3 mRNA are expressed in the kidney²¹. Immunohistochemistry showed that the two isoforms display a different pattern of distribution. Indeed, whereas Akt1 was predominantly found in tubules and almost undetectable in glomeruli, Akt2 was mainly located in podocytes (FIG. 1e). Double immunostaining using antibodies directed against WT1, a podocyte specific protein, confirmed this observation (FIG. 10. The distribution of the two proteins did not change after nephron reduction, except for the presence of a few Akt2-positive tubular cells (FIG. 1e).

### Akt2 Gene Inactivation Accelerates the Development of Glomerular Lesions

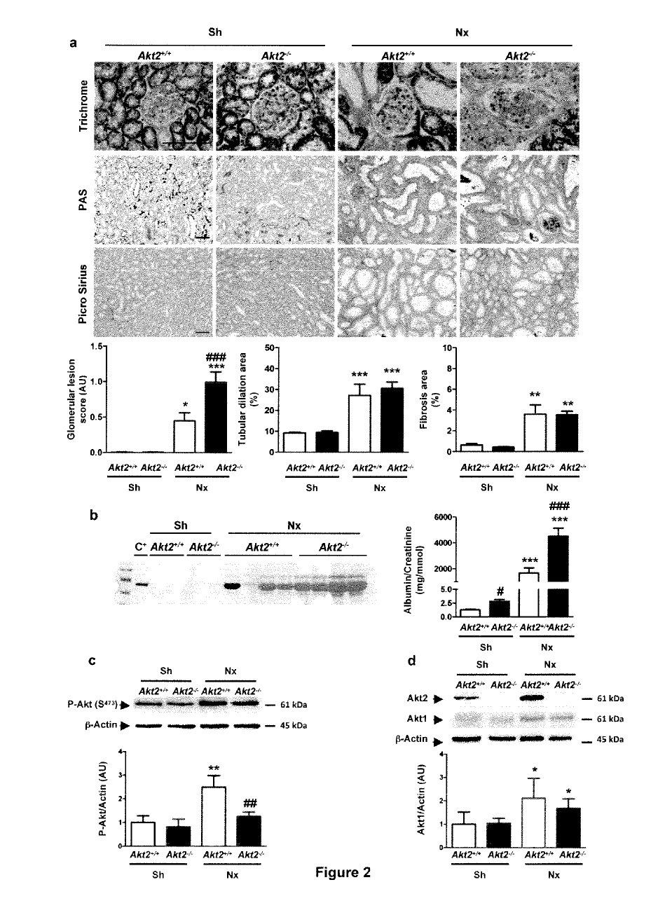

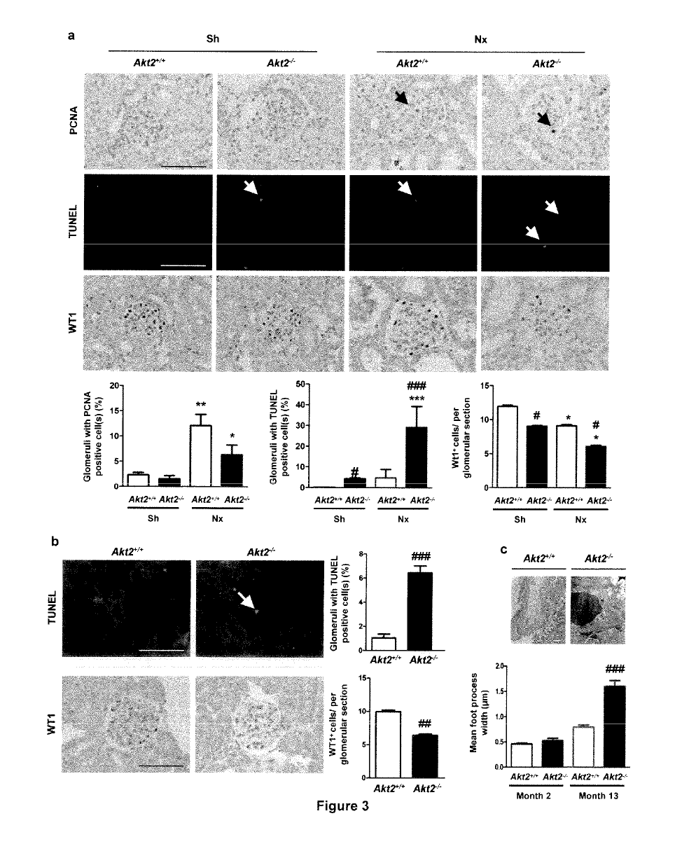

To investigate the role of Akt2 activation in CKD, we applied our experimental model of nephron reduction to Akt2^{-/-} mice. To this end, we first introduced the Akt2 mutated allele in the lesion-prone (FVB/N) background. The Akt2^{-/-} FVB/N mice reproduced normally and had no grossly apparent phenotype under physiological conditions (data not shown). As expected, two months after nephron reduction, wild-type mice developed renal lesions, mainly comprising of tubular dilations, interstitial fibrosis and mild glomerulosclerosis (FIG. 2a). The frequency and severity of glomerular lesions were dramatically increased in Akt2^{-/-} mice (FIG. 2a, see trichrome staining). In contrast, tubular and interstitial lesions were comparable regardless of the mouse genotype (FIG. 2a, see PAS and Picro Sirius staining, respectively). Quantification corroborated these observations and showed that Akt2^{-/-} mice had considerably higher glomerular, but similar tubular and interstitial lesion scores as compared with wild-type littermates (FIG. 2a). Consistently, albuminuria was significantly increased in Akt2^{-/-} mice (FIG. 2b). Western blot analysis on microdissected glomeruli showed that, P-Akt levels were similar between Akt2^{+/+} and Akt2^{-/-} sham-operated mice (FIG. 2c), indicating that Akt1 is sufficient to compensate for Akt2 gene inactivation under physiological conditions. However, after nephron reduction, Akt2 deficiency completely prevented the increase of Akt phosphorylation (Ser⁴⁷³) in glomeruli (FIG. 2c), despite the increase of Akt1 protein (FIG. 2d).

To rule out the possibility that the impact of Akt2 gene inactivation was only relevant in the subtotal nephrectomy model, we followed the evolution of kidney function in Akt2^{+/+} and Akt2^{-/-} mice from 2 to 13 months. Aging is known to be associated with progressive nephron reduction and late onset renal damage²². Notably, whereas urinary albumin excretion remained stable in Akt2^{+/+} mice throughout the 2- to 13-month follow-up, it progressively increased in Akt2^{-/-} mice (data not shown). Morphological analysis showed more severe glomerular lesions in 13-month old Akt2^{-/-} mice than in wild-type littermates (data not shown).

### Hypertension and Diabetes do not Account for Increased Glomerular Lesions in Akt2^{-/-} Mice

Since our results demonstrated that Akt2 is critical for glomerular function after nephron reduction, we next aimed at elucidating the mechanisms underlying the beneficial effect of Akt2 in CKD. Akt has been implicated in cardiovascular adaptation to hypertension and in diabetes^{23,24}, two major risk factors of CKD progression¹¹. Hence, we first analyzed the impact of Akt gene inactivation on these events. As expected, mean arterial blood pressure significantly increased in wild-type mice as compared to control animals two months after nephron reduction. However, the increase was of the same magnitude in Akt2^{-/-} mice (data not shown). Consistently, the hypertrophy of the media of renal large arteries was comparable in Akt2^{-/-} and Akt2^{+/+} mice (data not shown). In addition, the diameter of small renal arteries was identical between control and subtotally nephrectomized mice regardless of the genotype (data not shown). As previously described in other genetic backgrounds^{23,24}, sham-operated FVB/N Akt2^{-/-} mice displayed mild fasting hyperglycemia as compared to wild-type littermates (data not shown). Nephron reduction resulted in a weak increase of fasting glycemia in both Akt2^{+/+} and Akt2^{-/-} animals. Although the increase was slightly higher in Akt2^{-/-} mice (data not shown), neither diabetes mellitus nor glycosuria could be detected in mutant mice up to two months after nephron reduction (data not shown). Hence, whereas hypertension or diabetes do not seem to account for impaired glomerular lesions in our model, the inhibition of Akt pathway in podocytes strongly supports a cell autonomous effect.

### Akt2 Regulates Podocyte Survival and Morphology

It is known that the balance between cell proliferation and apoptosis as well as the maintenance of foot processes are critical events for podocyte adaptation to pathological conditions¹⁶. Previous studies have shown that Akt pathway activation may stimulate cell proliferation, inhibit apoptosis and control the ability of cells to migrate¹⁰. We therefore monitored the number and function of Akt2^{-/-} podocytes after nephron reduction. As expected, the rate of cell proliferation was very low in control mice; less than 5% of total glomeruli displayed at least one PCNA-positive cell (FIG. 3a). The number of PCNA-positive cells significantly increased after nephron reduction regardless of the genotype (FIG. 3a), although the increment tended to be lower in Akt2^{-/-} mice. Conversely, whereas the percentage of glomeruli with apoptotic cells was modestly enhanced in wild-type mice after nephron reduction, it dramatically increased in Akt2^{-/-} mice (FIG. 3a). A significant number of apoptotic cells were also observed in control Akt2^{-/-} mice (FIG. 3a). The increased apoptosis associated with lower cell proliferation correlated with podocyte rarefaction; in fact, the number of WT1-positive cells was 50% of the control wild-type value in Akt2^{-/-} nephrectomized mice (FIG. 3a). Similar results were obtained in aging Akt2^{-/-} mice, in which the development of albuminuria was also associated with increased glomerular apoptosis and podocytes rarefaction (FIG. 3b). Transmission electron microscopy revealed marked foot process effacement in 13-month old Akt2^{-/-} mice (FIG. 3c), indicating that podocyte rarefaction is associated with severe damage of the remaining ones. Notably, slight focal foot process effacement was detectable by three months of age in Akt2^{-/-} mice and led to the doubling of the width between foot processes in 13-month old animals (FIG. 3c).

Akt2 Regulates Critical Targets for Podocyte Survival after Nephron Reduction

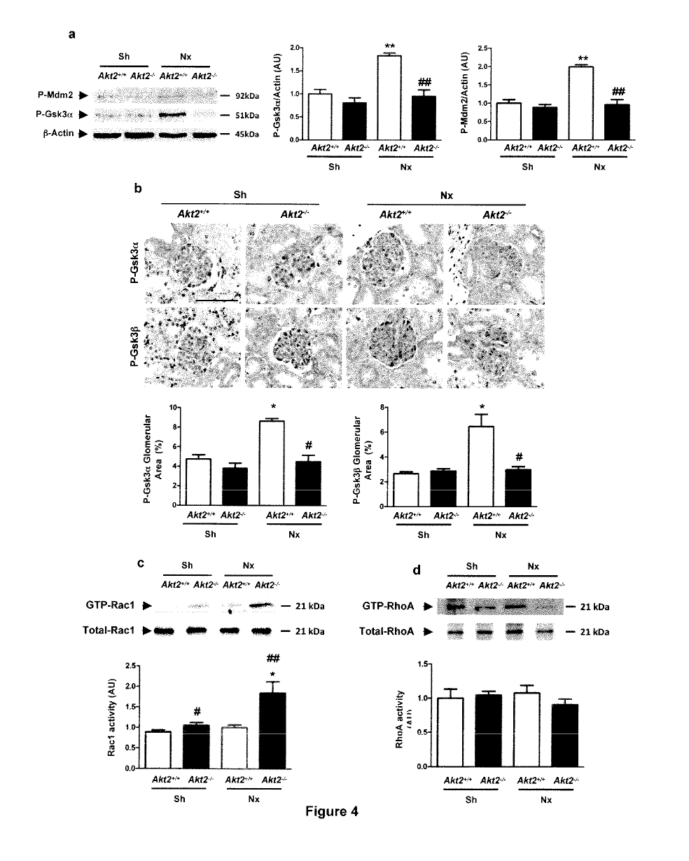

We next tried to identify the molecular link between Akt2 activation and podocyte modifications. Our data demonstrated that podocytes lacking Akt2 are more susceptible to apoptosis after nephron reduction. Akt prevents apoptosis by modulating both anti- and pro-apoptotic molecules, such as Mdm2 or Gsk3¹⁰. Western blot analysis revealed that the phosphorylation of both Mdm2 and Gsk3α was significantly increased in microdissected glomeruli of wild-type animals as compared to sham-operated animals. However, P-Mdm2 and P-Gsk3α levels did not change in glomeruli of Akt2 mutant mice two months after nephron reduction (FIG. 4a). Immunohistochemistry confirmed the absence of P-Gsk3α and P-Gsk3β up-regulation in podocytes of Akt2^{-/-} mice (FIG. 4b).

Our results showed that the remaining Akt2^{-/-} podocytes display ultrastructural changes that suggest cytoskeleton alterations. Rac1 is a small G protein belonging to the Rho family²⁵. By cycling between an active GTP-bound state and an inactive GDP-bound state, this protein family regulates many biological processes including cytoskeleton organization, cell cycle progression and apoptosis²⁵. Interestingly, excessive Rac1-GTP leads to foot process effacement, albuminuria and glomerulosclerosis in mice²⁶. We therefore hypothesized that Akt allows podocyte cytoskeleton to adapt to nephron reduction by controlling Rac1 activation. Using GST-Pak pull down assay, we observed that kidneys of sham-operated Akt2^{-/-} mice displayed a spontaneous higher amount of Rac1-GTP as compared to Akt2^{+/+} littermates (FIG. 4c). More importantly, whereas the amount of Rac1-GTP did not change after nephron reduction in Akt2^{+/+} mice, it markedly increased in Akt2^{-/-} mice. Since RhoA and Rac1 have been found to have opposite effects on the cytoskeleton²⁷, we also assessed RhoA-GTP activity. However, neither Akt2 deletion nor nephron reduction affected the amount of GTP bound RhoA, although the total amount tended to be lower in Akt2^{-/-} mice after nephron reduction (FIG. 4d). To provide further evidence for a role of Rac1, we measured Rac1-GTP during aging-induced nephropathy. Rac1 activity was also increased in damaged kidneys of 13-month old Akt2^{-/-} mice as compared with control littermates (data not shown).

### Akt Function in Podocyte is Isoform Specific

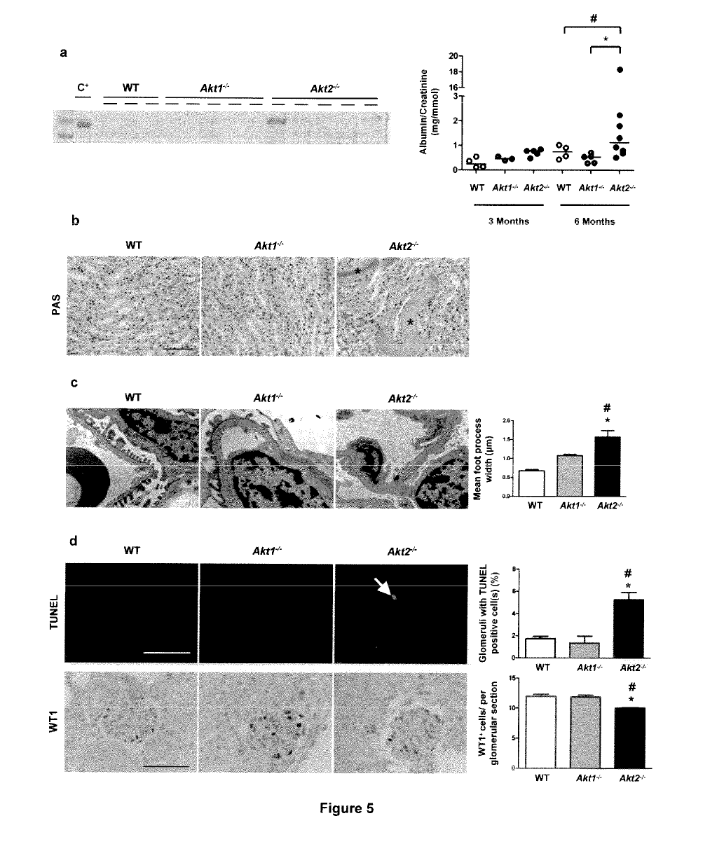

We observed that Akt2 is particularly enriched in podocytes where its expression is crucial for adaptation to nephron reduction. Recent studies have revealed that, despite their high sequence homology, Akt isoforms may regulate distinct physiological phenomena²⁸. In order to definitively rule out a role of Akt1 in podocyte homeostasis, we studied the evolution of kidney function in Akt1^{-/-} and Akt2^{-/-} mice from 2 to 6 months. Since inactivation of Akt1 gene was embryonic lethal in the lesion-prone FVB/N background (data not shown), we studied C57BL/6 Akt1^{-/-} and Akt2^{-/-} mice. Remarkably, the increase of urinary albumin excretion over time was observed exclusively in Akt2^{-/-} mice (FIG. 5a). Tubular casts, which are a morphological marker of proteinuria, were found only in kidneys of Akt2^{-/-} mice (FIG. 5b). More importantly, we observed that whereas Akt2^{-/-} podocytes displayed an effacement of foot processes and a decrease of cell survival, the morphology and the number of podocytes lacking Akt1 did not differ from those of wild-type littermates (FIG. 5c, d). Collectively, these data point to Akt2 as the critical isoform acting in glomerular pathophysiology.

### Podocyte Specific Akt2 Deletion Accelerates Podocyte Injury and Proteinuria

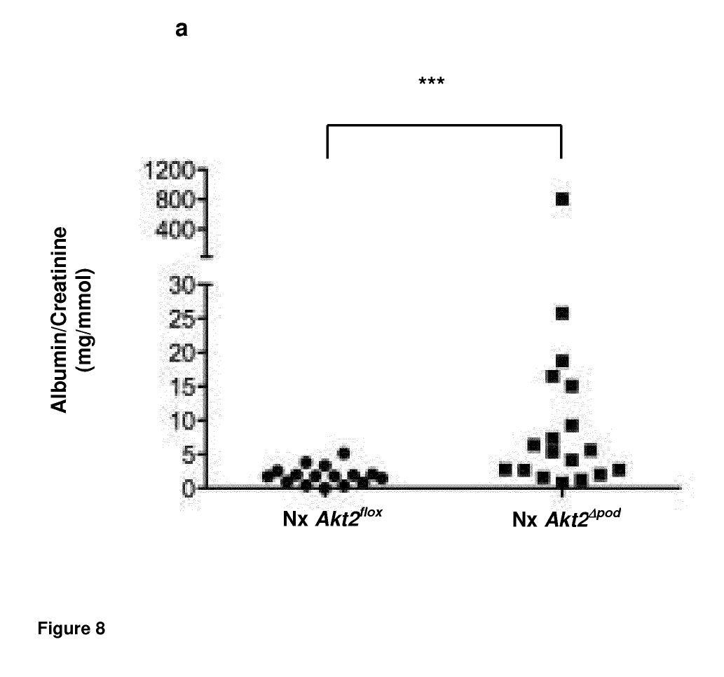

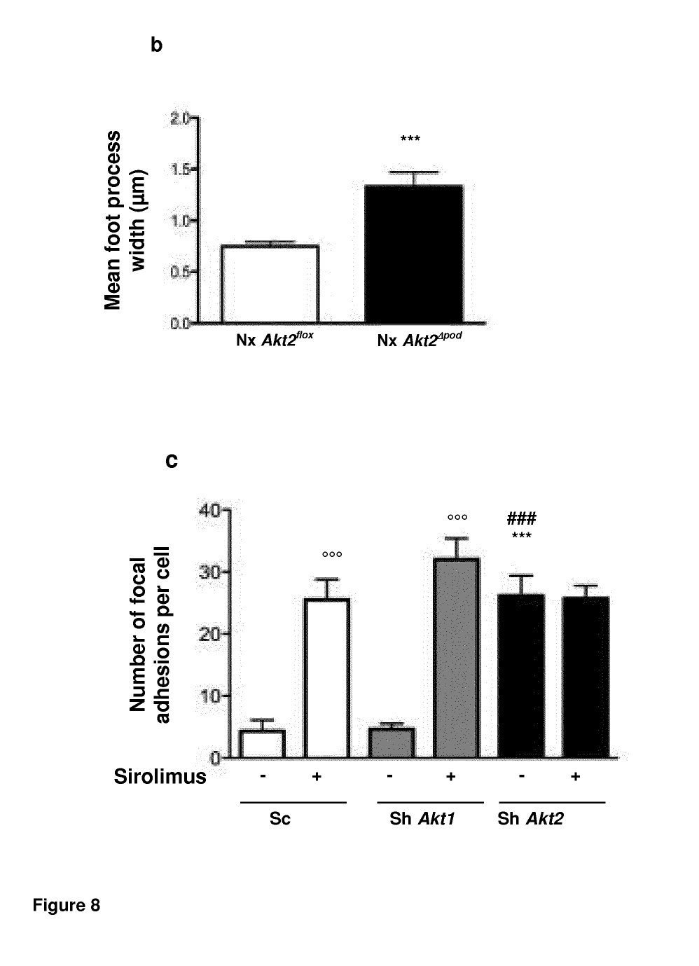

Finally, to unequivocally determine the cell autonomous function of Akt2 in podocytes, we generated podocyte-specific Akt2 mutant mice (Akt2^{pod}) by crossing mice bearing floxed Akt2 alleles (Akt2^{flox}) with a Nphs2.Cre deleter strain. Immunohistochemical analysis confirmed the marked reduction (90±2%) of Akt2 protein expression in podocytes of mutant mice (data not shown). Akt2^{pod} mice born in an expected Mendelian ratio and were indistinguishable from Akt2^{flox} littermates at least up to six months after birth under physiological conditions (data not shown). However, when subjected to subtotal nephrectomy, Akt2^{pod} mice developed significantly proteinuria (FIG. 8b). Consistently, we observed a marked effacement of foot processes (FIG. 8c), suggesting a direct effect of Akt2 on podocyte cytoskeleton. To further characterize the function played by Akt2 in podocyte cytoskeleton maintenance, we generated different podocyte cell lines using shRNA lentivirus targeting either Akt2 or Akt1 (data not shown). Phalloidin staining revealed that F-actin localized to the cell cortex, but also diffusely throughout the cell cytoplasm in control cells (data not shown). Akt2 knockdown resulted in a completed reorganization of F-actin cytoskeleton and massive formation of focal adhesion processes, as judged by the increased number of paxillin patches (data not shown, FIG. 8c) a phenotype reminiscent of that reported in Rictor silenced cells⁵⁸. In contrast, both the morphology of actin distribution and the pattern of paxillin staining were undistinguishable between in Akt1 knockdown and control cells (data not shown, FIG. 8c). Both phenotypes were confirmed with two independent ShRNA direct against distinct Akt1 and Akt2 sequences.

Akt Phosphorylation by mTORC2 is Critical to Prevent Proteinuria in Human CKD

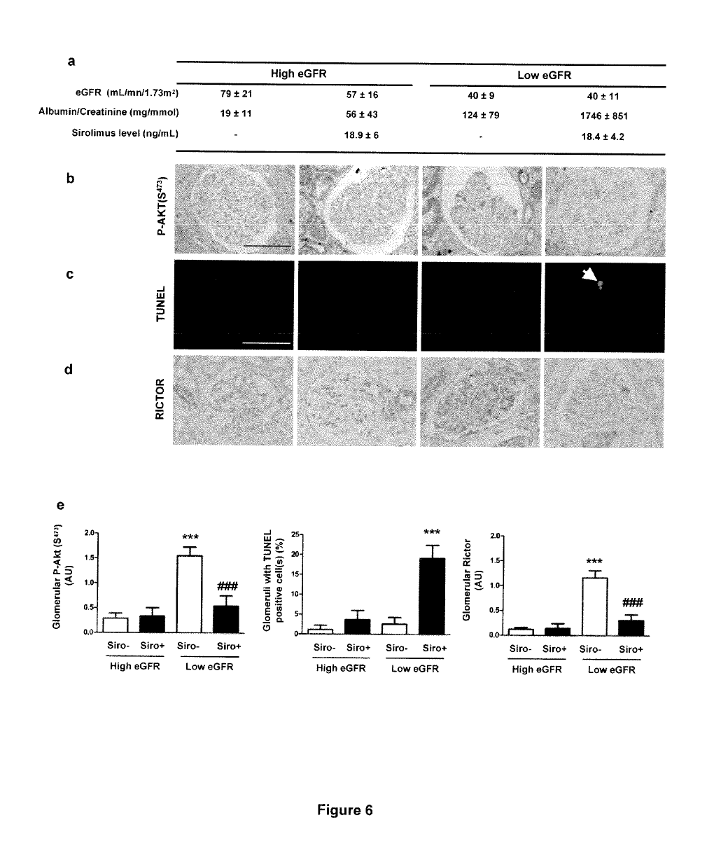

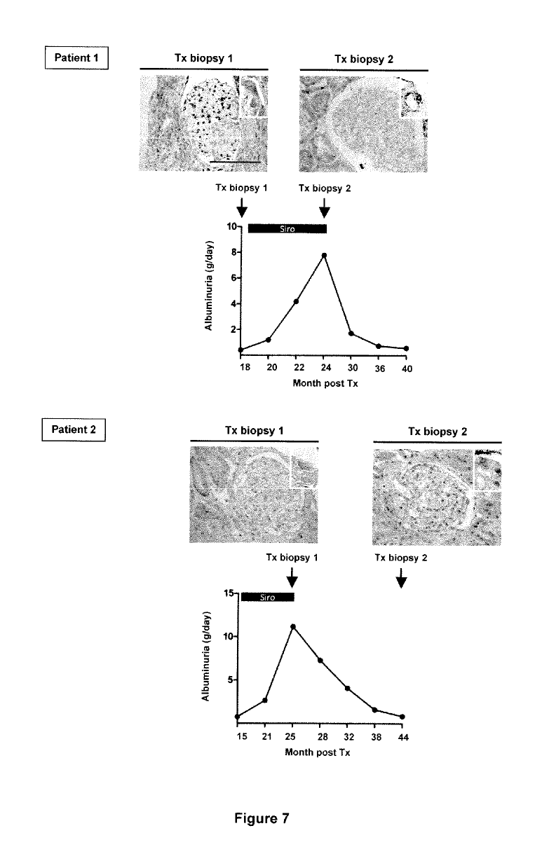

Sirolimus, a specific inhibitor of mammalian target of rapamycin (mTOR), is a potent immunosuppressive drug currently used after transplantation²⁹. mTOR is a component of two complexes, mTORC1 that mediates S6 kinase and 4EBP phosphorylation, and mTORC2 that regulates Akt Ser⁴⁷³ phosphorylation. Sirolimus is an allosteric inhibitor acting on mTORC1 activity. However, long-term treatment can also affect Akt phosphorylation in particular cell types via mTORC2³⁰. A number of studies have reported that, despite the fact sirolimus is not nephrotoxic, kidney transplant recipients might develop proteinuria and occasionally nephrotic syndrome upon sirolimus treatment³¹. This side effect is predominantly observed in patients who suffer from predamaged grafts (and, consequently, more severe nephron reduction), suggesting that, as in mice, Akt activation might be required for glomerular adaptation to nephron reduction. To test this hypothesis, we studied a cohort of kidney transplant recipients treated or not with sirolimus, displaying different stages of renal failure (FIG. 6a). As previously reported³¹, only patients with severe nephron reduction developed albuminuria upon sirolimus treatment (FIG. 6a). Remarkably, we observed that whereas P-Akt (Ser⁴⁷³) was barely detectable in transplant biopsies from patients with preserved renal function, a strong staining was found in podocytes of patients with severe nephron reduction (FIG. 6b, e). Colocalization experiments confirmed that Akt is mainly activated in podocytes (data not shown). In addition, we observed that, as in mice, Akt2 is predominantly expressed in podocytes, whereas Akt1 was mainly located in tubules (data not shown). Sirolimus administration prevented the activation of Akt in patients with impaired renal function (FIG. 6b, e) which was associated with increased glomerular apoptosis (FIG. 6c, e). More importantly, by studying P-Akt (Ser⁴⁷³) in sequential transplant biopsies, we showed a mechanistic association between the inhibition of Akt phosphorylation and the development of albuminuria (FIG. 7). In fact, whereas administration of sirolimus resulted in severe albuminuria and decreased Akt phosphorylation in the first patient (given as an example), its removal restored the phosphorylation of Akt at Ser⁴⁷³ and reversed albuminuria in the second patient (FIG. 7).

Since long-term sirolimus treatment may affect both mTORC1 and mTORC2 activities in vitro³⁰, we finally thought that it was important to determine which of these two pathways was involved in the proteinuria-promoting effect of sirolimus. Immunohistochemical analysis of two downstream targets of mTORC1 (S6RP and 4EBP) revealed that podocytes were only occasionally stained for P-S6RP and P-4EBP and that phosphorylation was not enhanced in kidneys of patients with compromised renal function (data not shown). As a consequence, the staining remained undetectable in sirolimus-treated patients (data not shown). In contrast, we observed that the expression of RICTOR, a critical component of mTORC2, was markedly increased in podocytes of patients with severe nephron reduction, and that sirolimus completely abolished such an increase, consistent with the inhibition of Akt phosphorylation on Ser⁴⁷³ (FIG. 6d, e). Collectively, these results suggest that mTORC2 is required to maintain Akt phosphorylation in stressed podocytes.

### Discussion

The molecular pathways that counteract the stress imposed by compensation following nephron reduction are largely unknown. By combining experimental models of nephron reduction with Akt1 and Akt2 knockout mice, we have uncovered a novel function for Akt proteins and showed that Akt2, but not Akt1, plays a critical role in podocyte adaptation to nephron reduction. Inactivation of the Akt2 gene resulted in podocyte apoptosis and foot process effacement after both experimental and aging-induced nephron reduction, which led to severe proteinuria and glomerulosclerosis. Furthermore, we have identified Gsk3, Mdm2 and Rac1 as potential intermediates between Akt2 activation and podocyte survival. More importantly, we have shown that these experimental observations were relevant to human diseases. In kidney transplant recipients with severe nephron reduction, treatment by sirolimus decreased the phosphorylation of Akt2 in podocytes resulting in marked proteinuria and podocyte loss. Surprisingly, the deleterious effect of sirolimus seemed to be mediated by mTORC2 rather than mTORC1 inhibition. These results unveil a novel molecular pathway of podocyte adaptation to stress and show that Akt2 acts as a survival factor whose activation might mark patients at risk to develop proteinuria after treatment with mTOR inhibitors.

Although many studies have been conducted to elucidate the molecular mechanisms leading to podocyte injury during CKD³²⁻³⁶, the signaling pathways that allow podocytes to compensate and survive after nephron loss, the hallmark of all CKD, are still unknown. Our study points to Akt as the kinase that prevents podocyte dedifferentiation and loss in this setting. In fact, we observed that Akt is activated in podocytes in response to nephron reduction, consistent with previous in vitro studies reporting an activation of Akt in cultured podocytes submitted to stress³⁷. More importantly, we demonstrated that the preclusion of such activation by Akt2 gene disruption or sirolimus treatment led to morphological and functional podocyte damage in both humans and mice. Although we did not use a podocyte specific invalidation strategy, we provide strong evidence for a cell autonomous effect of Akt2 in podocyte adaptation to nephron reduction. First, Akt2 displayed a strong and specific expression pattern in podocytes. Second, Akt2 deletion completely abrogated the activation of Akt as well as that of its down-stream signaling pathway in response to nephron reduction specifically in podocytes. Finally, Akt2 deletion did not induce significant systemic changes that could participate in glomerular injury, such as diabetes or hypertension. It is noteworthy that, consistent with its expression pattern, the absence of Akt2 did not affect the severity of tubulo-interstitial lesions, suggesting that specific molecular pathways are involved in glomerular and tubular adaptation.

Phenotypic analyses of Akt isoform knockout mice revealed both specific and redundant functions for each isoform^{21,23,38}. In this study, we found a striking dichotomy of Akt isoform distribution in the kidney with a preferential location of Akt2 to podocytes. More importantly, we demonstrated that this location is functionally relevant: Akt2^{-/-}, but not Akt1^{-/-} mice, displayed a glomerular phenotype. Intriguingly, although immunohistochemistry failed to detect significant amounts of Akt1 in glomeruli, western blot experiments clearly showed that this isoform is also expressed in glomeruli. It is likely that technical limitations account for these discrepancies. Although we cannot completely rule out the possibility that Akt1 has a function in Akt2^{-/-} glomeruli under physiological conditions, our data clearly showed that this isoform is not sufficient to compensate for the lack of Akt2 in glomerular homeostasis after nephron reduction.

One of the most prominent features of podocytes is the very specific organization of the cytoskeleton that appears crucial for their proper function³⁹. Indeed, a strong correlation has been recently shown between podocyte cytoskeleton modifications and podocyte-enhanced migratory properties and the development of albuminuria and glomerulosclerosis^{33,40,41}. Interestingly, the ability to regulate cytoskeleton organization and migration diverge dramatically between the Akt isoforms⁴². Akt1^{-/-} mouse embryonic fibroblasts have reduced migratory properties and prominent stress fibers, whereas those from Akt2^{-/-} mice have enhanced migratory properties and lamellipodia formation⁴². Consistently, we observed that Akt2^{-/-} kidneys displayed an up-regulation of Rac1, a key regulator of cell migration and lamellipodia formation. Loss of Rac1 inhibition has been recently shown to induce foot process effacement, albuminuria and glomerulosclerosis²⁶, suggesting that Rac1 activation in Akt2^{-/-} stressed podocytes might be mechanistically involved in their morphological changes. It should be noted that Akt2, but not Akt1, has been shown to inhibit Rac1 in mouse embryonic fibroblasts⁴². Hence, we propose that by its selective ability to control cytoskeleton organization, the Akt2 isoform has a specific function in podocyte biology.

Akt2 has also been shown to be the critical isoform mediating insulin receptor signaling in skeletal muscle and liver^{23,24}. A very recent study has shown that the insulin receptor is also expressed and acts in podocytes⁴³. Indeed, podocyte-specific insulin receptor inactivation resulted in podocyte loss and foot process effacement leading to progressive glomerular disease⁴³. This phenotype is highly reminiscent of what we observed in aging Akt2^{-/-} mice. Moreover, paralleling our observation, insulin receptor activation reduced Rac1 and enhanced Rho activity in cultured podocytes⁴³. Taking all these data together, it is tempting to speculate that Akt2 is the critical link between the insulin receptor and podocyte homeostasis. Since defective insulin signaling in podocytes is emerging as a critical determinant of diabetic nephropathy, Akt2 may represent a novel therapeutic target in this very common disease.

In the present study we also observed that Akt2 gene inactivation led to increased podocyte loss after nephron reduction by enhancing apoptosis. Akt is known to prevent apoptosis by inhibiting several actors involved in the control of cell death⁴⁴⁻⁴⁶. In particular, Mdm2, an E3 ubiquitin-ligase that modulates p53 stability, and Gsk-3, a serine/threonine kinase that controls caspase 3 activity, have been shown to be critical targets. Interestingly, we observed that Akt deficiency prevented both Mdm2 and Gsk-3 activation after nephron reduction, suggesting that the phosphorylation of these molecules is critical for podocyte survival after nephron reduction. On the other hand, the increase of Rac1 activity may be also involved in Akt2^{-/-} podocyte loss, since this GTPase has been shown to control apoptosis in several pathophysiological conditions^{25,47}. Interestingly, a recent study, using podocyte-specific inactivation of the Atg5 gene, has suggested that also autophagy may be involved in the maintenance of podocyte during aging⁴⁸. Although Akt/mTORC1 is usually considered as an inhibitor of autophagy⁴⁹, the similarity between Akt2^{-/-} and Atg5^{-/-} in aging mice raises the possibility of an interplay between Akt2 and autophagy in podocyte biology⁴⁸. Whether Akt2 is also involved in the control of autophagy in podocytes requires further investigations.

Sirolimus is known to induce albuminuria in renal transplant recipients with compromised renal function, but the mechanism involved is unknown⁵⁰. Our results strongly suggest that, by preventing the compensatory phosphorylation of Akt in podocytes, sirolimus led to albuminuria in patients with significant nephron reduction. Moreover, we discovered that mTORC2 rather than mTORC1 is the crucial target of sirolimus in this situation. So far, it has been accepted that sirolimus acts mainly by inhibiting mTORC1. Only a few in vitro studies have shown that prolonged exposure to the drug can lead to mTORC2 inhibition^{30,51}. Notably, our patients were treated by sirolimus for several months. Hence, our work is the first clear demonstration of a clinically significant effect of sirolimus on mTORC2. Because of the key role played by the Akt pathway in several pathological conditions, including cancer and cardiovascular disease, we can anticipate a growing use of Akt inhibitors in therapies⁵². In this regard, our results add, at least, two important contributions. First, we have identified a sensitive way to predict the adverse effect of sirolimus on renal function. Determining mTORC2/Akt2 activation in podocytes may identify patients with the highest risk to develop proteinuria. Second, since we showed that albuminuria is linked to mTORC2/Akt2 inhibition, we expect that specific TORC1 or Akt1 targeting therapies should be a valuable strategy to overcome this side effect in patients with reduced renal function.

In conclusion, this work has established mTORC2/Akt2 as a stress-responsive axis in podocyte biology that prevents cytoskeleton alteration and apoptosis after nephron reduction. Disruption of this adaptive pathway leads to glomerular lesions and albuminuria in both humans and mice. Therefore, Akt2 in podocytes may be both a prognostic marker to predict the deleterious proteinuric effect of mTOR inhibitors and as well as a therapeutic target for the maintenance of glomerular functions to prolong renal survival during chronic renal disease.

### REFERENCES

1. Heron, M., et al. Deaths: final data for 2006. Natl Vital Stat Rep 57, 1-134 (2009).
2. US Renal Data System, USRDS 2010 Annual Data Report: Atlas of Chronic Kidney Disease and End-Stage Renal Disease in the United States, National Institutes of Health, National Institute of Diabetes and Digestive and Kidney Diseases, Bethesda, Md. (2010).
3. Eknoyan, G., et al. The burden of kidney disease: improving global outcomes. Kidney Int 66, 1310-1314 (2004).
4. Go, A. S., Chertow, G. M., Fan, D., McCulloch, C. E. & Hsu, C. Y. Chronic kidney disease and the risks of death, cardiovascular events, and hospitalization. N Engl J Med 351, 1296-1305 (2004).
5. Hostetter, T. H. Progression of renal disease and renal hypertrophy. Annu Rev Physiol 57, 263-278 (1995).
6. Hostetter, T. H. Hyperfiltration and glomerulosclerosis. Semin Nephrol 23, 194-199 (2003).
7. Kriz, W. & LeHir, M. Pathways to nephron loss starting from glomerular diseases-insights from animal models. Kidney Int 67, 404-419 (2005).
Franke, T. F. PI3K/Akt: getting it right matters. Oncogene 27, 6473-6488 (2008).
9. Ceci, M., Ross, J., Jr. & Condorelli, G. Molecular determinants of the physiological adaptation to stress in the cardiomyocyte: a focus on AKT. Journal of molecular and cellular cardiology 37, 905-912 (2004).
10. Manning, B. D. & Cantley, L. C. AKT/PKB signaling: navigating downstream. Cell 129, 1261-1274 (2007).
11. Oudit, G. Y., et al. Loss of PTEN attenuates the development of pathological hypertrophy and heart failure in response to biomechanical stress. Cardiovasc Res 78, 505-514 (2008).
12. Wu, M., Falasca, M. & Blough, E. R. Akt/protein kinase B in skeletal muscle physiology and pathology. J Cell Physiol 226, 29-36.
13. Tejada, T., et al. Failure to phosphorylate AKT in podocytes from mice with early diabetic nephropathy promotes cell death. Kidney Int 73, 1385-1393 (2008).
14. Stylianou, K., et al. The PI3K/Akt/mTOR pathway is activated in murine lupus nephritis and downregulated by rapamycin. Nephrol Dial Transplant 26, 498-508.
15. Chuang, P. Y. & He, J. C. Signaling in regulation of podocyte phenotypes. Nephron Physiol 111, p 9-15 (2009).
16. Shankland, S. J. The podocyte's response to injury: role in proteinuria and glomerulosclerosis. Kidney Int 69, 2131-2147 (2006).
17. Mundel, P. & Reiser, J. Proteinuria: an enzymatic disease of the podocyte? Kidney Int 77, 571-580.
18. Zhu, J., et al. Nephrin mediates actin reorganization via phosphoinositide 3-kinase in podocytes. Kidney Int 73, 556-566 (2008).
19. Huber, T. B., et al. Nephrin and CD2AP associate with phosphoinositide 3-OH kinase and stimulate AKT-dependent signaling. Mol Cell Biol 23, 4917-4928 (2003).
20. Pillebout, E., et al. Proliferation and remodeling of the peritubular microcirculation after nephron reduction: association with the progression of renal lesions. Am J Pathol 159, 547-560 (2001).
21. Tschopp, O., et al. Essential role of protein kinase B gamma (PKB gamma/Akt3) in postnatal brain development but not in glucose homeostasis. Development 132, 2943-2954 (2005).
22. Yumura, W., et al. Age-associated changes in renal glomeruli of mice. Exp Gerontol 24, 237-249 (1989).
23. Cho, H., et al. Insulin resistance and a diabetes mellitus-like syndrome in mice lacking the protein kinase Akt2 (PKB beta). Science 292, 1728-1731 (2001).
24. Garofalo, R. S., et al. Severe diabetes, age-dependent loss of adipose tissue, and mild growth deficiency in mice lacking Akt2/PKB beta. J Clin Invest 112, 197-208 (2003).
25. Jaffe, A. B. & Hall, A. Rho GTPases: biochemistry and biology. Annu Rev Cell Dev Biol 21, 247-269 (2005).
26. Shibata, S., et al. Modification of mineralocorticoid receptor function by Rac1 GTPase: implication in proteinuric kidney disease. Nat Med 14, 1370-1376 (2008).
27. Sander, E. E., ten Klooster, J. P., van Delft, S., van der Kammen, R. A. & Collard, J. G. Rac downregulates Rho activity: reciprocal balance between both GTPases determines cellular morphology and migratory behavior. J Cell Biol 147, 1009-1022 (1999).
28. Gonzalez, E. & McGraw, T. E. The Akt kinases: isoform specificity in metabolism and cancer. Cell cycle (Georgetown, Tex. 8, 2502-2508 (2009).
29. Kreis, H., et al. Long-term benefits with sirolimus-based therapy after early cyclosporine withdrawal. J Am Soc Nephrol 15, 809-817 (2004).
30. Sarbassov, D. D., et al. Prolonged rapamycin treatment inhibits mTORC2 assembly and Akt/PKB. Mol Cell 22, 159-168 (2006).
31. Letavernier, E., Pe'raldi, M. N., Pariente, A., Morelon, E. & Legendre, C. Proteinuria following a switch from calcineurin inhibitors to sirolimus. Transplantation 80, 1198-1203 (2005).
32. Niranjan, T., et al. The Notch pathway in podocytes plays a role in the development of glomerular disease. Nat Med 14, 290-298 (2008).
33. Wei, C., et al. Modification of kidney barrier function by the urokinase receptor. Nat Med 14, 55-63 (2008).
34. Reiser, J., et al. Induction of B7-1 in podocytes is associated with nephrotic syndrome. J Clin Invest 113, 1390-1397 (2004).
35. Ma, H., et al. Inhibition of podocyte FAK protects against proteinuria and foot process effacement. J Am Soc Nephrol 21, 1145-1156.
36. Dai, C., et al. Wnt/beta-catenin signaling promotes podocyte dysfunction and albuminuria. J Am Soc Nephrol 20, 1997-2008 (2009).
37. Faour, W. H., Thibodeau, J. F. & Kennedy, C. R. Mechanical stretch and prostaglandin E2 modulate critical signaling pathways in mouse podocytes. Cell Signal 22, 1222-1230.
38. Cho, H., Thorvaldsen, J. L., Chu, Q., Feng, F. & Birnbaum, M. J. Akt1/PKBalpha is required for normal growth but dispensable for maintenance of glucose homeostasis in mice. J Biol Chem 276, 38349-38352 (2001).
39. Faul, C., Asanuma, K., Yanagida-Asanuma, E., Kim, K. & Mundel, P. Actin up: regulation of podocyte structure and function by components of the actin cytoskeleton. Trends Cell Biol 17, 428-437 (2007).
40. Reiser, J., et al. Podocyte migration during nephrotic syndrome requires a coordinated interplay between cathepsin L and alpha3 integrin. J Biol Chem 279, 34827-34832 (2004).
41. Yanagida-Asanuma, E., et al. Synaptopodin protects against proteinuria by disrupting Cdc42:IRSp53:Mena signaling complexes in kidney podocytes. Am J Pathol 171, 415-427 (2007).
42. Zhou, G. L., et al. Opposing roles for Akt1 and Akt2 in Rac/Pak signaling and cell migration. J Biol Chem 281, 36443-36453 (2006).
43. Welsh, G. I., et al. Insulin signaling to the glomerular podocyte is critical for normal kidney function. Cell Metab 12, 329-340.
44. Jope, R. S. & Johnson, G. V. The glamour and gloom of glycogen synthase kinase-3. Trends Biochem Sci 29, 95-102 (2004).
45. Gottlieb, T. M., Leal, J. F., Seger, R., Taya, Y. & Oren, M. Cross-talk between Akt, p53 and Mdm2: possible implications for the regulation of apoptosis. Oncogene 21, 1299-1303 (2002).
46. Zhou, B. P., et al. HER-2/neu induces p53 ubiquitination via Akt-mediated MDM2 phosphorylation. Nat Cell Biol 3, 973-982 (2001).
47. Shen, E., et al. Rac1 is required for cardiomyocyte apoptosis during hyperglycemia. Diabetes 58, 2386-2395 (2009).
48. Hartleben, B., et al. Autophagy influences glomerular disease susceptibility and maintains podocyte homeostasis in aging mice. J Clin Invest 120, 1084-1096.
49. Jung, C. H., Ro, S. H., Cao, J., Otto, N. M. & Kim, D. H. mTOR regulation of autophagy. FEBS Lett 584, 1287-1295.
50. Letavernier, E. & Legendre, C. mToR inhibitors-induced proteinuria: mechanisms, significance, and management. Transplantation reviews (Orlando, Fla. 22, 125-130 (2008).
51. Vollenbroker, B., et al. mTOR regulates expression of slit diaphragm proteins and cytoskeleton structure in podocytes. Am J Physiol Renal Physiol 296, F418-426 (2009).
52. Lindsley, C. W., Barnett, S. F., Layton, M. E. & Bilodeau, M. T. The PI3K/Akt pathway: recent progress in the development of ATP-competitive and allosteric Akt kinase inhibitors. Curr Cancer Drug Targets 8, 7-18 (2008).
53. Terzi, F., et al. Targeted expression of a dominant-negative EGF-R in the kidney reduces tubulo-interstitial lesions after renal injury. J Clin Invest 106, 225-234 (2000).
54. Viau, A., et al. Lipocalin 2 is essential for chronic kidney disease progression in mice and humans. J Clin Invest 120, 4065-4076 (2010).
55. Mollet, G., et al. Podocin inactivation in mature kidneys causes focal segmental glomerulosclerosis and nephrotic syndrome. J Am Soc Nephrol 20, 2181-2189 (2009).
56. Lautrette, A., et al. Angiotensin II and EGF receptor cross-talk in chronic kidney diseases: a new therapeutic approach. Nat Med 11, 867-874 (2005).
57. Shankland, S. J., Pippin, J. W., Reiser, J. & Mundel, P. Podocytes in culture: past, present, and future. Kidney international 72, 26-36 (2007).
58. Sarbassov, D. D., et al. Rictor, a novel binding partner of mTOR, defines a rapamycin-insensitive and raptor-independent pathway that regulates the cytoskeleton. Curr Biol 14, 1296-1302 (2004).

1. An in vitro method for predicting the toxicity of a treatment with mTOR inhibitors in a patient in need of such treatment, said method comprising the steps of:
a. providing a tissue sample from a patient to be tested; and,
b. determining, in said tissue sample, the activation level of Akt2 pathway.

2. The method according to claim 1, wherein activation of Akt2 pathway is indicative of a likelihood of toxicity of a treatment with mTOR inhibitors in said patient.

3. The method according to claim 1, wherein said tissue sample is a biopsy from kidney tissue.

4. The method according to claim 1, wherein said activation of Akt2 pathway is determined by detecting of P-Akt2 (Ser⁴⁷³) protein or Rictor protein.

5. The method according to claim 4, wherein detection of phosphorylated P-Akt2 (Ser⁴⁷³) protein or Rictor protein in a corresponding control sample is indicative of activation of Akt2 pathway as compared to no or a barely detectable amount in a control tissue sample.

6. The method according to claim 1, wherein said toxicity is proteinuria.

7. The method according to claim 1, wherein said patient is a transplant patient in need of a treatment for preventing transplant rejection.

8. The method according to claim 1, wherein said patient is a patient in need of a treatment for preventing kidney transplant rejection.

9. A kit for predicting the toxicity of a treatment with mTOR inhibitors in a patient in need thereof by determining, in a tissue sample from the patient, the activation level of Akt2 pathway, the kit, comprising at least a binding reagent specific of P-Akt2 (Ser⁴⁷³) protein and labels for detecting the amount of binding reagent.

10. A method for treating a subject suffering from a kidney disorder and in need of an immunosuppressant treatment, comprising administering to said subject a therapeutically effective amount of a selective mTORC1 or Akt1 inhibitor.

11. The method of claim 10, wherein said mTORC1 or Akt1 inhibitor is a compound that inhibits mTORC1 kinase activity with an IC50 at least 10 times lower than the IC50 measured for mTORC2 kinase activity.

12. The method according to claim 10, wherein said mTORC1 or Akt1 inhibitor is a compound that inhibits mTORC1 kinase but exhibits no significant inhibition of mTORC2 kinase.

13. A method for treating a patient in need of mTOR inhibitor comprising: requesting a test providing the results of an analysis to determine whether the patient has a normal, low or high level of activation of Akt2 pathway and administering: either,
(i) a treatment with mTOR inhibitor if said patient has a normal or low level of activation of Akt2 pathway; or,
(ii) a treatment with an immunosuppressant drug that does not target the mTOR pathway if said patient has a high level of activation of Akt2 pathway.

14. The method according to claim 13, wherein said mTOR inhibitor is selected from the group consisting of: AZD-8055, OSI-027, INK-128, WYE-132, Torin1, PP242, PP30, Ku-0063794, AP-23573, TAFA93, biolimus-7, biolimus-9, sirolimus, deforolimus, temsirolimus, everolimus, rapamycin and their derivatives or pharmaceutically acceptable salts.

15. The kit of claim 9, further comprising one or more of buffers, controls, and instructions for use of the kit.

16. The method according to claim 1, further comprising treating the patient with no or a low level of activation of Akt2 pathway with a mTOR inhibitor treatment.

17. The method according to claim 1, further comprising treating the patient with a high level of activation of Akt2 pathway with an immunosuppressant drug that does not target the mTOR pathway.

18. The method of claim 1, wherein determination of the activation level of Akt2 pathway includes detection of the phosphorylated Akt2 protein level using an in situ immunohistochemical method on the tissue sample.

19. The method of claim 18, wherein an antibody specific for P-Akt2 (Ser⁴⁷³) is used for the detection of the phosphorylated Akt2 protein level in the tissue sample.
